(19) United States
(12) Patent Application Publication
(10) Pub. No.: US 2026/0081018 A1
(43) Pub. Date: Mar. 19, 2026

(54) SYSTEM AND METHOD FOR ENHANCED HEALTHCARE DIAGNOSTICS USING NATURAL INTELLIGENCE

(71) Applicant: AI Atlas Inc., Hamilton (CA)

(72) Inventors: Mahdi Naghshvarianjahromi, Hamilton (CA); Shiva Kumar, Hamilton (CA); M. Jamal Deen, Hamilton (CA)

(21) Appl. No.: 18/885,314

(22) Filed: Sep. 13, 2024

(51) Int. Cl. G16H 50/20

(52) U.S. Cl. CPC G16H 50/20

(57) ABSTRACT

What is disclosed is: A system for natural intelligence (NI) processing for an intelligent health transponder comprising: A perceptor subsystem coupled to an executive subsystem, wherein: the perceptor subsystem comprises a posterior storage and one or more posterior processing modules. The executive subsystem comprises an executive storage, one or more executive processing modules and a planning module. A feedback subsystem is coupled to the executive subsystem, a transmission subsystem, a reception subsystem and a training dataset. The feedback subsystem comprises a feedback processing module, a feedback subsystem database, and a feedback processing module with a feedback subsystem firmware running on a feedback subsystem processor. An adaptive feedback path control module is coupled to the executive subsystem and the perceptor subsystem.

### FIELD OF THE INVENTION

The present disclosure relates to the field of healthcare, specifically to the application of natural intelligence (NI) to healthcare.

### BRIEF SUMMARY

A system for natural intelligence (NI) processing for an intelligent health transponder comprising: a perceptor subsystem communicatively coupled to an executive subsystem via interconnections, wherein: the perceptor subsystem comprises a posterior storage and one or more posterior processing modules coupled to each other by perceptor subsystem interconnections, and the executive subsystem comprises an executive storage, one or more executive processing modules and a planning module coupled to each other by executive subsystem interconnections; a feedback subsystem communicatively coupled to the executive subsystem, a transmission subsystem, a reception subsystem and a training dataset, wherein: the feedback subsystem comprises a feedback processing module communicatively coupled to a feedback subsystem database, further wherein: the feedback processing module comprises a feedback subsystem firmware running on a feedback subsystem processor; an adaptive feedback path control module communicatively coupled to the executive subsystem and the perceptor subsystem, wherein: the perceptor subsystem receives perceptions comprising a plurality of generated output symbols, the perceptor subsystem receives a plurality of representative input symbols, based on the received plurality of generated output symbols and plurality of representative input symbols, the one or more posterior processing modules determining whether a suitable posterior model is available in the posterior storage, when a suitable posterior model is available, the one or more posterior processing modules retrieves a posterior model from the posterior storage, and the one or more posterior processing modules communicates the retrieved posterior model to the adaptive feedback path control module, the adaptive feedback path control module estimates a diagnostic error (DE) using the retrieved posterior model, the adaptive feedback path control module communicates the estimated DE to the executive subsystem, when the estimated DE is below a threshold, the planning module selects a prospective action from the executive storage, at least one of the planning module and the one or more executive processing modules test the selected prospective action in a virtual environment, at least one of the planning module and the one or more executive processing modules determines whether the selected prospective action is beneficial, when the selected prospective action is beneficial, either the planning module or the one or more executive processing modules communicates signals comprising the selected prospective action to the feedback subsystem, and the feedback processing module: receives the signals comprising the selected prospective action, determines, based on the received signals, an adjustment to implement the selected prospective action, and transmits signals to perform the determined adjustment to one or more components within the transmission or the reception, or the training dataset.

The foregoing and additional aspects and embodiments of the present disclosure will be apparent to those of ordinary skill in the art in view of the detailed description of various embodiments and/or aspects, which is made with reference to the drawings, a brief description of which is provided next.

### BRIEF DESCRIPTION OF THE DRAWINGS

The foregoing and other advantages of the disclosure will become apparent upon reading the following detailed description and upon reference to the drawings.

FIG. 1A illustrates an example embodiment of an intelligent healthcare transponder system.

FIG. 1B illustrates an example embodiment of a healthcare transmission.

FIG. 1C illustrates an example embodiment of a representative signal modulator within a multidimensional modulator.

FIG. 1D illustrates an example embodiment of an orthogonal modulator within a multidimensional modulator.

FIG. 1E illustrates an example embodiment of a healthcare reception.

FIG. 1F illustrates an example embodiment of an orthogonal demodulator within a correlation receiver.

FIG. 1G illustrates an example embodiment of a representative signal demodulator within a correlation receiver.

FIG. 1H illustrates an example embodiment of a healthcare feedback subsystem.

FIG. 1I illustrates an example embodiment of a transmission sequence for an intelligent health transponder.

FIG. 1J illustrates an example embodiment of a reception sequence for an intelligent health transponder.

FIG. 2A illustrates an example embodiment of a natural intelligence processing subsystem.

FIG. 2B illustrates an example embodiment of a perceptor subsystem.

FIG. 2C illustrates an example embodiment of an executive subsystem.

FIG. 3 illustrates an example embodiment of a posterior processing flow.

FIG. 4A illustrates an example embodiment of a posterior extraction processing flow.

FIG. 4B illustrates an example embodiment of a 2-dimensional space for received symbols.

FIG. 4C illustrates an example embodiment of a process to determine discretization parameters.

FIG. 5 illustrates an example embodiment of a process to calculate health capacity.

FIG. 6A illustrates an example embodiment a process following a training dataset update.

FIG. 6B illustrates an example embodiment of a process to resume steady state operation.

FIG. 6C illustrates an example embodiment of a process related to an increase in reference rate.

FIG. 7A illustrates part of an example embodiment of an executive subsystem process flow.

FIG. 7B illustrates part of an example embodiment of an executive subsystem process flow.

FIG. 8 illustrates an example embodiment of adjustments performed by feedback subsystem firmware.

FIGS. 9-13 illustrate example embodiments of representative signals.

FIGS. 14-18 illustrate example embodiments of representative input symbols.

While the present disclosure is susceptible to various modifications and alternative forms, specific embodiments or implementations have been shown by way of example in the drawings and will be described in detail herein. It should be understood, however, that the disclosure is not intended to be limited to the particular forms disclosed. Rather, the disclosure is to cover all modifications, equivalents, and alternatives falling within the spirit and scope of an invention as defined by the appended claims.

### BACKGROUND

The rapid advancements in healthcare technology have underscored the critical need for smart systems capable of autonomously managing and diagnosing health conditions in real-time. Traditional machine learning (ML) and artificial intelligence (AI) approaches in healthcare face significant challenges, including the handling of non-Gaussian and non-linear data distributions, the impact of defective or unbalanced datasets, and the susceptibility to noise and cyber-attacks. These factors can severely compromise the reliability and efficiency of diagnostic processes, leading to inaccurate or delayed diagnoses, which are particularly detrimental or even fatal in critical healthcare settings.

Furthermore, in current ML applications in healthcare, determining the maximum number of health conditions that can be reliably diagnosed while minimizing errors is a significant challenge. Most systems aim to improve accuracy without considering the fundamental limits on the number of conditions that can be diagnosed accurately.

Additionally, many ML applications in healthcare require feature extraction before operation. This limitation hampers the ability of these applications to function effectively with defective datasets, as feature extraction must occur beforehand. This can be problematic if the data is noisy or incomplete. The requirement for feature extraction may reduce efficiency in real-time applications, as the system requires pre-processing of features, which delays the diagnostic process. If features are incorrectly extracted, diagnostic errors may arise.

To address these challenges, there has been a paradigm shift towards using advanced cognitive systems, inspired by natural intelligence (NI). NI systems draw inspiration from the cognitive functions of the human brain, employing a perception-action cycle (PAC) that dynamically interacts with the environment to make informed decisions. These systems are designed to be adaptive, resilient, and capable of handling the complexities inherent in healthcare data, particularly under challenging conditions such as data corruption or cyber threats.

Prior art in this domain have explored various applications of NI in healthcare but with notable limitations. For example, M. Naghshvarianjahromi, S. Kumar and M. J. Deen, “Brain-Inspired Intelligence for Real-Time Health Situation Understanding in Smart e-Health Home Applications,” in IEEE Access, vol. 7, pp. 180106-180126, 2019, hereinafter referred to as “Naghshvarianjahromi 1”, focuses on diagnosing diseases but does not contemplate the calculation of healthcare capacity and is not fully automatic due to the necessity of feature extraction before operation.

M. Naghshvarianjahromi, S. Majumder, S. Kumar, N. Naghshvarianjahromi and M. J. Deen, “Natural Brain-Inspired Intelligence for Screening in Healthcare Applications,” in IEEE Access, vol. 9, pp. 67957-67973, 2021, hereinafter referred to as “Naghshvarianjahromi 2”, discusses a system designed for screening purposes with multi-actions and multi-observables but similarly lacks healthcare capacity calculations and automation in feature extraction.

Both Naghshvarianjahromi 1 and Naghshvarianjahromi 2 then suffer from the previously discussed disadvantages due to the lack of healthcare capacity calculation and automation in feature extraction.

### DETAILED DESCRIPTION

One of ordinary skill in the art would appreciate that while the systems and methods detailed below target healthcare; these systems and methods could be applied to other artificial intelligence (AI) and machine learning (ML) systems where evaluation of training dataset capacity is required.

The systems and methods detailed below address the shortcomings mentioned above. In particular, the systems and methods detailed below include techniques to calculate healthcare capacity, inspired by techniques from information and communication theory to calculate channel capacity.

Unlike traditional healthcare systems that rely on feature extraction and selection, the systems and methods disclosed below bypasses these steps, enabling a more efficient diagnostic process even in the presence of defective datasets or cyber-attacks.

The systems and methods discussed below detail the deployment of a transmission and reception model for healthcare data sets using an intelligent healthcare transponder. The transmission and reception model facilitates the rigorous analysis and management of healthcare scenarios by applying principles of communication and information theory to the healthcare domain. By treating health conditions as inputs and diagnostic decisions as outputs, the systems and methods disclosed below mimic the data transmission process in a communication channel, optimizing the diagnostic accuracy and reliability based on the calculated healthcare capacity. In the model below, input signals which are representative of a

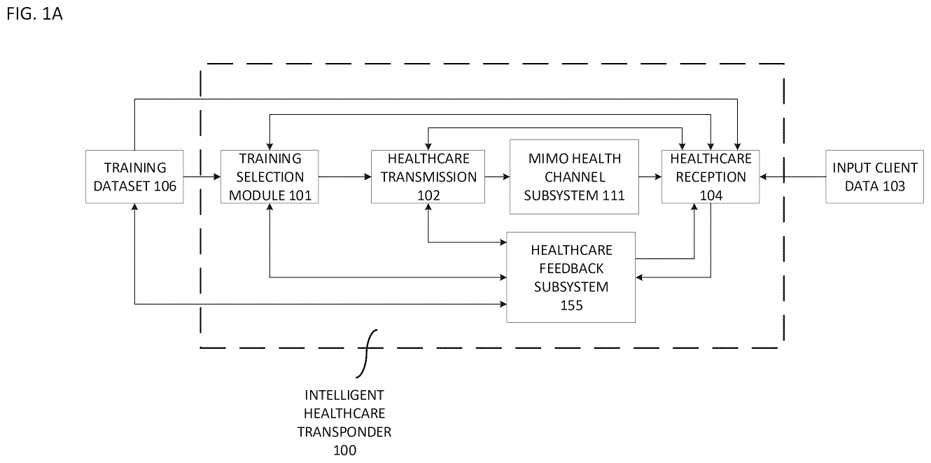

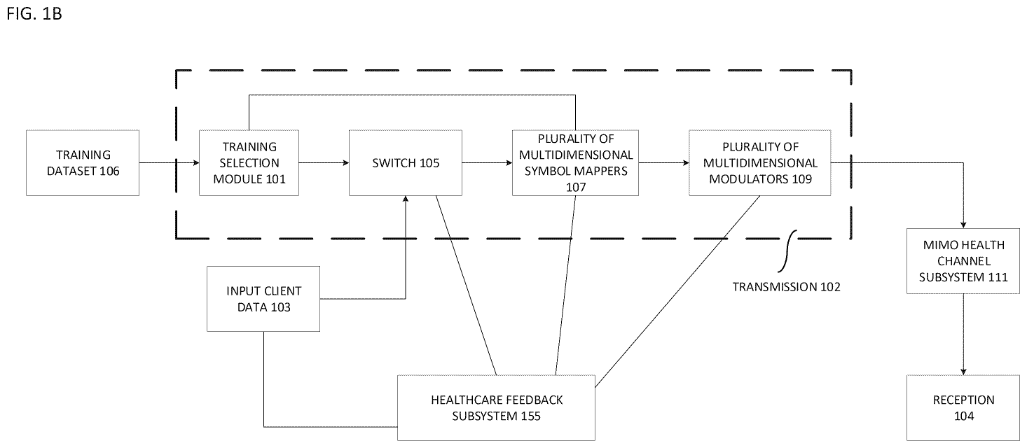

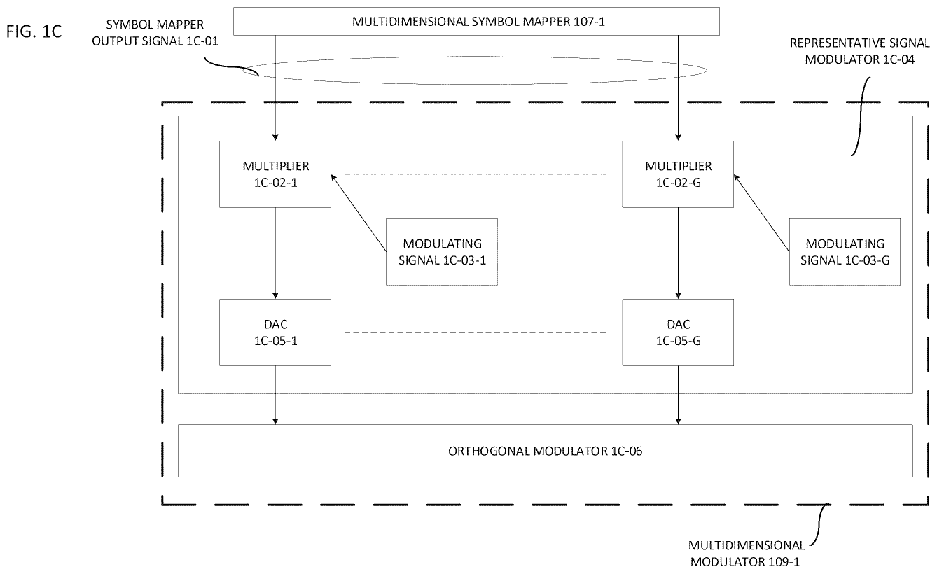

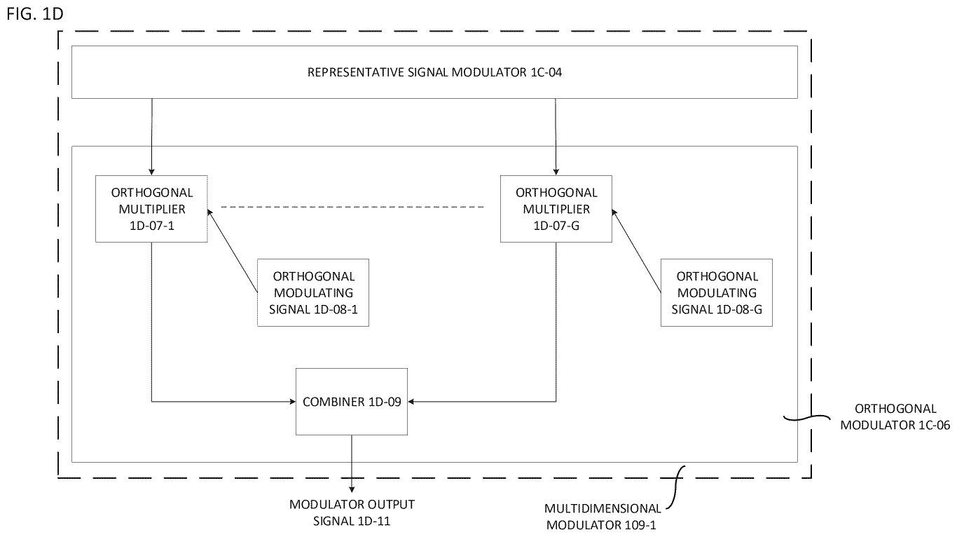

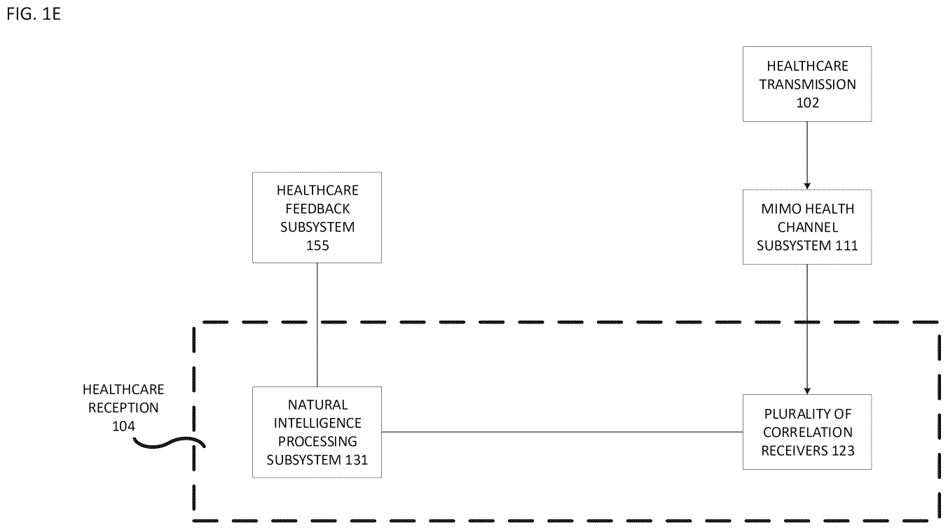

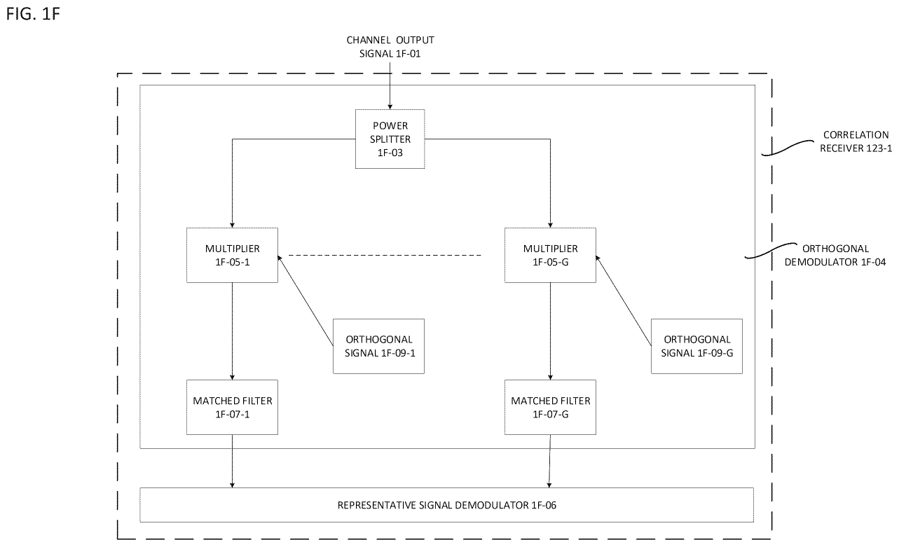

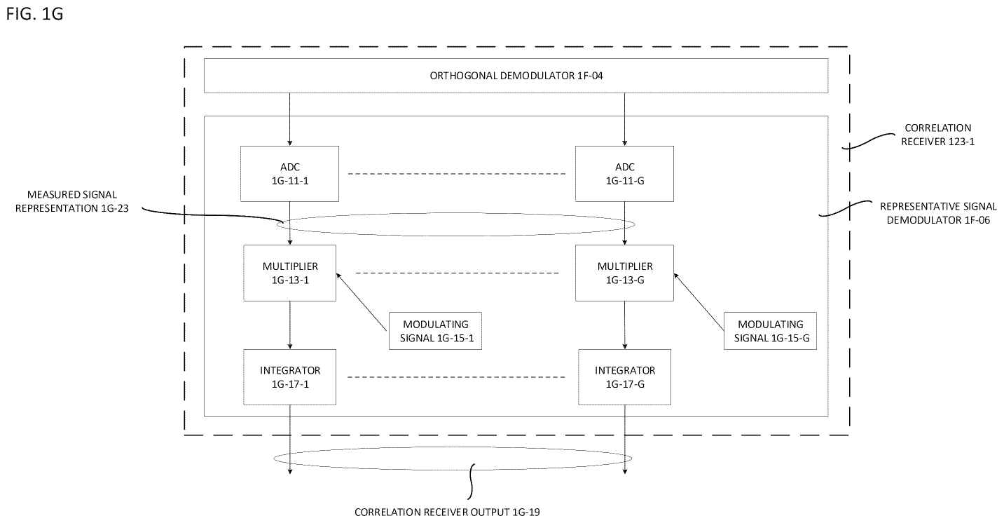

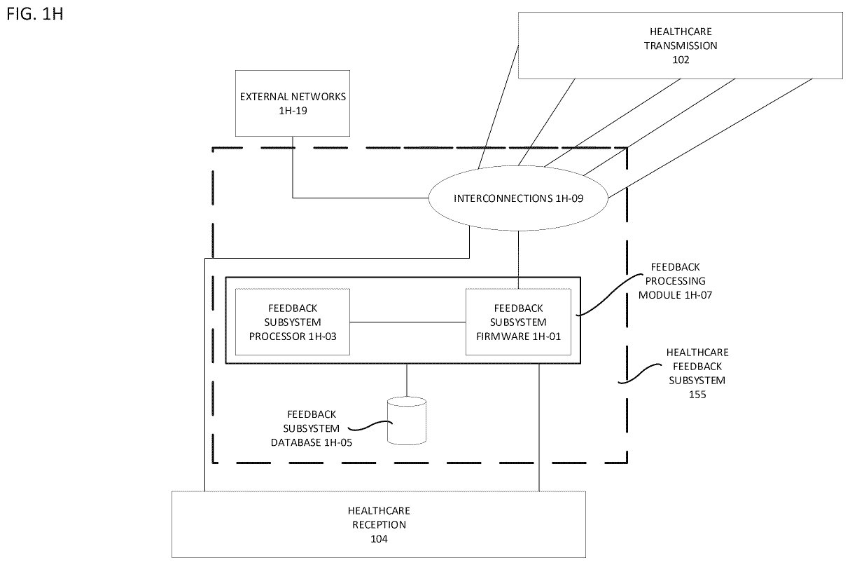

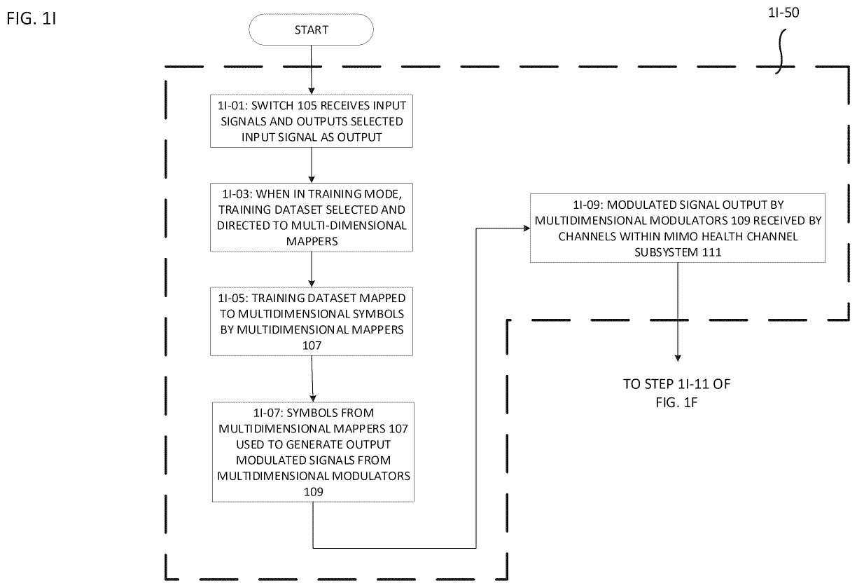

FIG. 1A illustrates an example embodiment of an intelligent healthcare transponder system 100 which integrates natural intelligence (NI). As is discussed below, this system enables the deployment of an approach to model healthcare scenarios using a MIMO health channel subsystem.

Intelligent healthcare transponder system 100 comprises training selection module 101, healthcare transmission 102, MIMO health channel subsystem 111, healthcare reception 104, and healthcare feedback subsystem 155. In some embodiments, these components are interconnected with each other using communication techniques known to those of ordinary skill in the art. These components and the operation of these components are discussed below.

Intelligent healthcare transponder system 100 can be implemented in a variety of ways. In some embodiments, intelligent healthcare transponder system 100 is implemented in hardware. In yet other embodiments, intelligent healthcare transponder system 100 is implemented in software. In yet other embodiments, intelligent healthcare transponder system 100 is implemented on a chip. In yet other embodiments, intelligent healthcare transponder system 100 is implemented using a cloud-based implementation. In yet other embodiments, intelligent healthcare transponder system 100 is implemented using one or more servers.

Intelligent healthcare transponder storage 171 plays the role of storing data, programs and commands to be used by the components of intelligent healthcare transponder system 100. As shown in FIG. 1A, intelligent healthcare transponder storage 171 is communicatively coupled to the other components of intelligent healthcare transponder 100. Intelligent healthcare transponder storage 171 is implemented using storage and memory techniques known to those of ordinary skill in the art.

FIG. 1B shows an example embodiment of healthcare transmission 102. The operation of healthcare transmission 102 is discussed in conjunction with FIG. 1I.

FIGS. 1I and 1J shows an example embodiment of a process flow in intelligent healthcare transponder system 100 for the training process using training dataset 106 from receipt to diagnosis. FIG. 1I shows the transmission sequence 1I-50, and FIG. 1J shows a reception sequence 1I-52.

Importantly, steps 1I-01, 1I-03, 1I-05, 1I-07, 1I-09, 1I-11 and 1I-13 of FIGS. 1I and 1J model the transformation of representative signals from a training dataset into measured signals via transmission over a MIMO health channel subsystem. This modeling approach enables the use of known techniques from information theory to calculate channel capacity in the calculation of health capacity of a training data set. The prior art does not contemplate such a modeling approach. As is discussed below, the calculation of health capacity enables various actions to be performed within the intelligent healthcare transponder system 100, including:
improving the performance of the intelligent healthcare transponder system 100;
alerting users to potential cyber-attacks on intelligent healthcare transponder system 100;
alerting users to potential data entry mistakes in the training dataset 106; and
detecting suspicious results in academic and other publications for a training dataset.

Furthermore, the results obtained from this modeling approach are in agreement with reported results for a well-known training dataset.

Referring to FIG. 1B in conjunction with FIG. 1E: healthcare transmission 102 comprises a plurality of health transmission inputs. Then, one or more of the plurality of health transmission inputs receives data from the training dataset 106.

Training dataset 106 comprises data from a number of classes, denoted as D, wherein each class comprises a plurality of data points from a number of sensors, denoted as L. Each class corresponds to a health condition or health situation. In the remainder of this specification, the term “number of classes” and D are used interchangeably. Also, in the remainder of this specification, the term “number of sensors” and L are used interchangeably. In some embodiments, training dataset 106 comprises one or more annotations, wherein the annotations comprise labels. An example of a label is “normal”.

An example of a training dataset 106 is the MIT-BIH Arrhythmia Database, hereinafter referred to as the “MIT database”. The MIT database comprises D=18 classes. The data collection utilizes L=2 sensors, corresponding to two electrocardiogram (ECG) leads.

In some embodiments, training dataset 106 is stored in intelligent healthcare transponder storage 171, and is retrieved as necessary.

Healthcare transmission 102 comprises training selection module 101. Training selection module 101 performs the role of selecting one or more data points representative of each of the D classes. One of ordinary skill in the art would appreciate that there are a variety of ways to select one or more data points representative of each of the D classes. For example, in some embodiments, the one or more data points which are representative of a class are selected randomly based on an annotation associated with the data points. For example, in some embodiments the representative one or more data points for a class comprises a signal randomly chosen from the signals annotated with the label “normal” or “typical” for that class. This random selection process is aimed at ensuring that each representative one or more data points captures the characteristic features of its respective class while minimizing biases. Performing this random selection for all the classes ensures comprehensive coverage of all health situations in the training dataset 106.

In some embodiments, the one or more data points comprise a representative signal. This representative signal is hereinafter referred to as

$${φ}_{h}^{g} ( t )$$

where h represents a channel number drawn from the set {1, 2, . . . , H} and g represents a dimension drawn from the set {1, 2, . . . , G}. In some embodiments, the representative signal has a duration T. Processes to determine the total number of channels H and total number of dimensions G are explained below.

Training selection module 101 can be implemented in a number of ways. In some embodiments, training selection module 101 is implemented in software. In some embodiments, training selection module 101 is implemented in hardware. In some embodiments, training selection module 101 is implemented in hardware and software. Training selection module 101 is also communicatively coupled to plurality of multidimensional modulators 109.

Then, in step 1I-01 of FIG. 1I: when the intelligent healthcare transponder system 100 is placed in training mode, which is explained below, one or more data points representative of each of the D classes are selected by training selection module 101, and directed to the inputs to switch 105. As explained above, the selected one or more data points comprise the previously described representative signal

$${φ}_{h}^{g} ( t ) .$$

The representative signals

$${φ}_{h}^{g} ( t )$$

are then transmitted to plurality of multidimensional modulators 109.

Switch 105 has a plurality of switch inputs and a plurality of switch outputs. Each of the plurality of switch inputs is communicatively coupled to the outputs from training selection module 101. Some of the plurality of switch outputs are communicatively coupled to the plurality of multidimensional symbol mappers 107. The number of switch outputs communicatively coupled to the plurality of multidimensional symbol mappers is determined based on L and D as described below.

In some embodiments, a number of switch outputs are made active based on L and D. This enables reconfiguration based on the L and D parameters of different training datasets. The determination of the number of switch outputs made active is described below.

Switch 105 can be implemented in a number of ways. In some embodiments, switch 105 is implemented in software. In some embodiments, switch 105 is implemented in hardware. In some embodiments, switch 105 is implemented in hardware and software.

In step 1I-03 of FIG. 1I: switch 105 receives the representative signals

$${φ}_{h}^{g} ( t )$$

as inputs and makes a determination whether to output the received input signals as output signals. When the intelligent healthcare transponder system 100 is in training mode, switch 105 outputs the received input signals as output signals. As will be explained below, in some embodiments a signal is sent to the intelligent healthcare transponder system 100 to enter steady state mode or training mode. In these embodiments, a signal is sent to switch 105 to indicate that the intelligent healthcare transponder system 100 is to enter steady state or training mode.

Each of the plurality of multi-dimensional symbol mappers 107 comprises an input and an output. The input to each of the plurality of multi-dimensional symbol mappers 107 is communicatively coupled to a switch output from the plurality of switch outputs.

Then, in step 1I-05 of FIG. 1I: each of the plurality of multi-dimensional symbol mappers 107 maps the representative one or more data points received from outputs of switch 105 to produce symbols, wherein each symbol has a plurality of symbol mapper dimensions. An example of this operation are demonstrated below for embodiments where the representative one or more data points is a representative signal

$${φ}_{h}^{g} ( t ) .$$

The number of multidimensional symbol mappers 107 corresponds to the total number of channels H, and the number of symbol mapper dimensions corresponds to the total number of dimensions G. H and G are determined based on L and D by setting G to the smaller of L and D; and H to the other. Then:
when L<D: H is set to D, and G is set to L. Since one switch 105 output is communicatively coupled to the input of one symbol mapper, then there are D switch 105 outputs connected to the D multidimensional symbol mappers. In the embodiments where a number of switch 105 outputs are made active, then D switch outputs are made active.
when L≥D: H is set to L, and G is set to D. There are L switch 105 outputs connected to the L multidimensional symbol mappers. In the embodiments where a number of switch 105 outputs are made active, then L switch 105 outputs are made active.

Setting the number of symbol mapper dimensions to the smaller of L and D results in reduced complexity.

For example, for the MIT database, since L=2 and D=18, then there are 18 multidimensional symbol mappers, each producing symbols with 2 symbol mapper dimensions. Then, G=2, and H=18.

In some embodiments, the plurality of multi-dimensional symbol mappers 107 is selected from a set of multi-dimensional symbol mappers and activated. The number which is activated depends on L and D. For example, for a training dataset where L<D, then D multidimensional symbol mappers are activated out of the set of multidimensional symbol mappers. Each of the activated D multidimensional symbol mappers are then configured to produce L-dimensional symbols. This enables reconfiguration based on the L and D parameters of different training datasets.

For multi-dimensional symbol mapper h the coordinates for class d and dimension g is given by:

$${x}_{h d}^{g} = {max}_{τ} \{ \frac{{\int }_{0}^{T} {φ}_{h}^{g} ( t ) {φ}_{d}^{g} ( t - τ ) d t}{\sqrt{{\int }_{0}^{T} {φ}_{h}^{g} ( t ) d t} \sqrt{{\int }_{0}^{T} {φ}_{d}^{g} ( t ) d t}} \} , \qquad ( Equation 1 )$$

where

$${φ}_{h}^{g} ( t )$$

is the previously described representative signals
h is drawn from the set {1, 2, . . . . H},
d is drawn from the set {1, 2, . . . . D},
g is drawn from the set {1, 2, . . . . G}, and
τ is a variable delay optimized to maximize the numerator.

Then, for each class d drawn from the set {1, 2, . . . , D} and channel h drawn from the set {1, 2, . . . . H}, there is a corresponding G-dimensional vector:

$${X}_{h d} = [ \begin{matrix}{x}_{hd}^{1} \\ {x}_{hd}^{2} \\ ⋮ \\ {x}_{hd}^{G}\end{matrix} ] , 1 \leq h \leq H , d ∈ \{ 1 , 2 , … , D \} , \qquad ( Equation 2 )$$

The G-dimensional signal coordinates and the constellation for channel h can be expressed as:

$${\hat{X}}_{n}^{k , h} ∈ \{ {X}_{h 1} , {X}_{h 2} , … , {X}_{hD} \} . \qquad ( Equation 3 )$$

where X_{hd} represents the co-ordinates for channel or symbol mapper h and class d, and

$${\hat{X}}_{n}^{k , h}$$

represents representative input symbols for the nth interval for perception action cycle (PAC) k and symbol mapper h.

In some embodiments, each X_{hd} is output sequentially from symbol mapper h. For example, Xₕ₁ is output from symbol mapper h, followed by Xₕ₂, then Xₕ₃ and so on until X_{hD}. Techniques to achieve such sequential output are known to those of ordinary skill in the art and are not discussed in detail here.

In some embodiments, the representative input symbols

$${\hat{X}}_{n}^{k , h}$$

are transmitted to, for example, intelligent healthcare transponder storage 171 to be used by other components of intelligent healthcare transponder system 100. In yet other embodiments, the representative input symbols

$${\hat{X}}_{n}^{k , h}$$

are transmitted to the other components of intelligent healthcare transponder system 100 for use in those components. For example, the representative input symbols

$${\hat{X}}_{n}^{k , h}$$

are transmitted to the healthcare reception 104 for use in natural intelligence processing subsystem 131, as explained below.

The plurality of multi-dimensional symbol mappers 107 can be implemented in a number of ways. In some embodiments, plurality of multi-dimensional symbol mappers 107 is implemented in software. In some embodiments, plurality of multi-dimensional symbol mappers 107 is implemented in hardware. In some embodiments, plurality of multi-dimensional symbol mappers 107 is implemented in hardware and software.

In some of the embodiments where there are hardware or hardware and software implementations; and the plurality of multi-dimensional symbol mappers 107 are selected from an available set of multi-dimensional symbol mappers and activated, there are power savings when the remainder are made inactive or put into sleep mode.

In yet other embodiments, the plurality of multi-dimensional symbol mappers 107 are implemented using pipelined or multi-threaded architectures.

The plurality of multi-dimensional symbol mappers 107 is communicatively coupled to a plurality of multi-dimensional modulators 109. Each of the plurality of multidimensional modulators 109 comprises an input and an output. Each of the plurality of multidimensional modulators produces output signals having a plurality of modulator dimensions. Then, each output of the plurality of multi-dimensional symbol mappers 107 is communicatively coupled to an input of each of the plurality of multi-dimensional modulators 109.

The number of multi-dimensional modulators 109 is equal to H, and the number of modulator dimensions is set to G. The determination of H and G have been discussed above.

Then, in step 1I-07 of FIG. 1I, for each class d drawn from the set {1, 2, . . . . D}: Each symbol mapper outputs a symbol comprising the corresponding G-dimensional vector X_{hd} produced in Equation 2, which is then input to a corresponding modulator. In some embodiments, this is performed sequentially, as described above.

Then, each of the H multi-dimensional modulators 109 receives a multi-dimensional symbol produced by the corresponding coupled multi-dimensional symbol mapper. Based on the received multi-dimensional symbol, each of the plurality of multi-dimensional modulators 109 produces an output modulated signal having the same number of modulator dimensions as the received multi-dimensional symbol. As explained previously, the representative signals

$${φ}_{g}^{h} ( t )$$

are transmitted by training selector module 101 upon selection in step 1I-01.

FIGS. 1C and 1D show these operations, for multi-dimensional modulator h=1 of the H multi-dimensional modulators, denoted as multi-dimensional modulator 109-1. Multi-dimensional modulator 109-1 is coupled to multi-dimensional symbol mapper 107-1.

Multi-dimensional modulator 109-1 comprises a representative signal modulator 1C-04 and an orthogonal modulator 1C-06. Representative signal modulator 1C-04 and the operations performed therein are now described with reference to FIG. 1C.

Multi-dimensional symbol mapper 107-1 produces symbol mapper output signal 1C-01 for class d=1, denoted by X₁₁, which is a G-dimensional vector.

Each element

$${x}_{n , 1}^{k , g}$$

of this G-dimensional vector then modulates a representative signal

$${φ}_{g}^{h} ( t - nT ) ;$$

where T is the signal duration and n is the current instant. The resulting modulated signal

$${s}_{Tx}^{k , h , g} ( t )$$

is the transmitted signal for PAC number k for channel h and dimension g, and is given by:

$${s}_{Tx}^{k , h , g} ( t ) = {x}_{n , h}^{k , g} {φ}_{g}^{h} ( t - nT ) \qquad ( Equation 4 )$$

Referring now to FIG. 1C: there are G multipliers 1C-02-1 to 1C-02-G in representative signal modulator 1C-04. Each of these multipliers correspond to a modulator dimension. The multipliers 1C-02-1 to 1C-02-G have associated modulating signals 1C-03-1 to 1C-03-G. These modulating signals are the representative signals

$${φ}_{g}^{h} ( t - nT ) .$$

For example, modulating signal 1C-03-1 in FIG. 1C is

$${φ}_{1}^{1} ( t - nT ) .$$

The output from multiplier 1C-02-1 is given by:

$${s}_{Tx}^{k , 1 , 1} ( t ) = {x}_{n , 1}^{k , 1} {φ}_{1}^{1} ( t - nT ) \qquad ( Equation 5 )$$

Similarly, modulating signal 1C-03-G in FIG. 1C which is

$${φ}_{G}^{1} ( t - nT )$$

is modulated by element

$${x}_{n , 1}^{k , G}$$

of the G-dimensional vector to produce output:

$${s}_{T x}^{k , 1 , G} ( t ) = {x}_{n , 1}^{k , G} {φ}_{G}^{1} ( t - n T ) \qquad ( Equation 6 )$$

The modulated signals then undergo digital-to-analog conversion within the G digital-to-analog converters (DACs) 1C-05-1 to 1C-05-G. The output from DAC g for modulator number h is given by

$${m}_{h}^{k , g} ( t )$$

and is sent to the orthogonal modulator which is part of the multi-dimensional modulator h. In the case of multi-dimensional modulator 109-1, the output from each DAC in representative signal modulator 1C-04 is then sent to orthogonal modulator 1C-06.

Each of these outputs are then used to modulate a corresponding one of the orthogonal modulating time-domain signals

$${f}_{g}^{h} ( t )$$

in the orthogonal modulator corresponding to channel number h and dimension g to produce a signal

$${m}_{h}^{k , g} ( t ) {f}_{g}^{h} ( t ) .$$

Referring to FIG. 1D now: In the case of multi-dimensional modulator 109-1, the orthogonal modulating time-domain signals 1D-08-1 to 1D-08-G, denoted as

$${f}_{g}^{1} ( t ) ,$$

are used to modulate the outputs

$${m}_{1}^{k , g} ( t ) {f}_{g}^{1} ( t )$$

in the orthogonal multipliers 1D-07-1 to 1D-07-G.

These signals are then combined to produce a signal that is transmitted through channel h of a multi-input multi-output (MIMO) health channel subsystem 111, given by:

$${M}_{h}^{k} ( t ) = {\sum }_{g = 1}^{G} {m}_{h}^{k , g} ( t ) {f}_{g}^{h} ( t )$$

Referring to FIG. 1D now: the signals are combined in the combiner 1E-09 to produce the signal:

$${M}_{1}^{k} ( t ) = {\sum }_{g = 1}^{G} {m}_{1}^{k , g} ( t ) {f}_{g}^{1} ( t ) \qquad ( Equation 8 )$$

Then, each of the H multidimensional modulators produces a G-dimensional output signal for one of the H channels in the MIMO health channel subsystem 111. The operations described above and in Equations (4)-(7) are then repeated for each of the D classes.

In some embodiments, the H multi-dimensional modulators 109 are selected from a set of multi-dimensional modulators and activated. Each of the activated H multi-dimensional modulators are then configured to produce G-dimensional modulated signals. This enables reconfiguration based on the L and D parameters of different training datasets. Techniques to achieve these steps are known to those of ordinary skill in the art and are not discussed in detail here.

Plurality of multi-dimensional modulators 109 can be implemented in a number of ways. In some embodiments, plurality of multi-dimensional modulators 109 is implemented in software. In some embodiments, plurality of multi-dimensional modulators 109 is implemented in hardware. In some embodiments, plurality of multi-dimensional modulators 109 is implemented in hardware and software.

In some of the embodiments where there are hardware or hardware and software implementations; and the plurality of multi-dimensional modulators 109 are selected from a set of available multi-dimensional modulators and activated, there are power savings when the remainder are made inactive or put into sleep mode.

MIMO health channel 111 has H channels, and each channel is used to transmit a G-dimensional symbol. Each channel in MIMO health channel 111 has an input and an output. Each of the plurality of channel inputs is communicatively coupled to the output of a corresponding one of the plurality of multi-dimensional modulators 109. Also, each of the plurality of channel inputs serves as an output from healthcare transmission 102. Then, in step 1I-09 of FIG. 1I: the modulated G-dimensional signal output by each of the H multi-dimensional modulators 109 is received by the input of the corresponding coupled channel.

Referring now to FIG. 1E: Each of the plurality of channel outputs from MIMO health channel subsystem 111 is coupled to healthcare reception 104. FIG. 1E also shows healthcare reception 104 in more detail. Healthcare reception 104 comprises natural intelligence processing subsystem 131 and plurality of correlation receivers 123. These will now be discussed in more detail, in conjunction with FIG. 1J.

Specifically, each of the plurality of channel subsystem 111 outputs is communicatively coupled to one of a corresponding plurality of correlation receivers 123. Each of the plurality of correlation receivers 123 comprises an input and an output. Then, each of the plurality of channel outputs is communicatively coupled to an input to one of the plurality of correlation receivers 123. The inputs to the plurality of correlation receivers then serve as inputs to the healthcare reception 104.

The correlation receiver for channel h utilizes a multi-dimensional correlator at the receiver to extract the signal vector

$${Y}_{n , h}^{k}$$

for each channel h, denoted as

$${Y}_{n , h}^{k} = [ {y}_{n , h}^{k , 1} , {y}_{n , h}^{k , 2} , … , {y}_{n , h}^{k , G} ] .$$

This is discussed below with respect to channel h=1.

FIGS. 1F and 1G illustrate one of the plurality of correlation receivers 123 for channel h=1 denoted as 123-1.

The output signal

$${v}_{h}^{k , g} ( t )$$

for each dimension g of channel h is then processed through matched filters and correlators as part of the demodulation process. In the presence of a non-Gaussian, non-linear environment (NGNLE), this process is represented as:

$${v}_{h}^{k , g} ( t ) = {z}_{h} ( {m}_{h}^{k , g} ( t ) , {w}_{h}^{g} ( t ) ) \qquad ( Equation 9 )$$

where

$${w}_{h}^{g} ( t )$$

represents the associated non-Gaussian noise (e.g., interference or distortion) for each dimension g and channel h,

$${m}_{h}^{k , g} ( t )$$

represents the transmitted signal for each channel h and dimension g,
zₕ(⋅) is a non-linear function of channel h dependent on the transmitted signal, the non-gaussian noise

$${w}_{h}^{g} ( t ) ,$$

and other system parameters.

In the case of channel number h=1.

$${v}_{1}^{k , g} ( t ) = {z}_{1} ( {m}_{1}^{k , g} ( t ) , {w}_{1}^{g} ( t ) )$$

The combined received signal

$${V}_{h}^{k} ( t )$$

for each channel h is denoted as:

$${V}_{h}^{k} ( t ) = \sqrt{G} {\sum }_{g = 1}^{G} {v}_{h}^{k , g} ( t ) {f}_{g}^{h} ( t ) \qquad ( Equation 10 )$$

In this case, the term \sqrt{G} represents power-based scaling.

In the case of channel number h=1:

$${V}_{1}^{k} ( t ) = \sqrt{G} {\sum }_{g = 1}^{G} {v}_{1}^{k , g} ( t ) {f}_{g}^{1} ( t ) \qquad ( Equation 11 )$$

This combined signal is received at the input to the correlation receiver h corresponding to channel number h. It is then passed through an equal power splitter which evenly divides the signal across the dimensions.

Referring to FIGS. 1F and 1J: In step 1I-11, for channel number h=1, the signal

$${V}_{1}^{k} ( t )$$

is received at the input to correlation receiver 123-1 and evenly divided across the dimensions by power splitter 1F-03.

In step 1I-13, a process to obtain a measured signal representation using the output from step 1I-11 is detailed.

As part of step 1I-13, for each dimension within correlation receiver h, the received signal is multiplied by an orthogonal function within an orthogonal multiplier corresponding to that dimension. One of ordinary skill in the art would know that the orthogonal function corresponds to the ones used in the plurality of multidimensional modulators 109.

Referring to FIG. 1F: for dimension g=1, the received signal is multiplied by orthogonal signal 1F-09-01 within multiplier 1F-05-1. Each orthogonal signal is represented by

$${f}_{g}^{h} ( t ) ,$$

which has been described previously. In the case of correlation receiver 123-1, each orthogonal signal is referred to as

$${f}_{g}^{1} ( t ) .$$

Step 1I-13 also comprises the following: for each dimension, the output from the multiplier for that dimension is processed through one of the matched filters for that dimension to extract

$${v}_{h}^{k , g} ( t ) .$$

As is known to one of ordinary skill in the art, in a matched filter, the received signal is correlated with a known template or reference signal, maximizing the signal-to-noise ratio for optimal detection. This process enables accurate extraction of

$${v}_{h}^{k , g} ( t ) .$$

The signal

$${v}_{h}^{k , g} ( t )$$

is then transmitted to the representative signal demodulator for correlation receiver h.

Referring to FIG. 1F: For example, for g=1, the output from multiplier 1G-05-1 is processed through matched filter 1G-07-1 to extract

$${v}_{1}^{k , 1} ( t ) .$$

The signal

$${v}_{1}^{k , 1} ( t )$$

is then transmitted to the representative signal demodulator 1F-06 for correlation receiver h=1.

Once within the representative signal demodulator, the output

$${v}_{h}^{k , g} ( t )$$

from each matched filter is passed through a corresponding Analog-to-Digital Converter (ADC). The output signal from the ADC is denoted as measured signal representation 1G-23, and is a representation of the measured signal from the sensors for each dimension g and channel h, denoted as

$${s}_{R x}^{k , h , g} ( t ) .$$

Referring now to FIG. 1G: For example, for g=1, the output

$${v}_{1}^{k , 1} ( t )$$

is passed through ADC 1G-11-1 to obtain a measured signal

$${s}_{R x}^{k , 1 , 1} ( t ) .$$

The waveform for dimensions 1 and 2 are denoted as

$${s}_{R x}^{k , 1 , 1} ( t ) and {s}_{R x}^{k , 1 , 2} ( t )$$

respectively.

The above-described process comprising steps 1I-01, 1I-03, 1I-05, 1I-07, 1I-09, 1I-11 and 1I-13 of FIGS. 1I and 1J models the transformation of representative signals into measured signals for each dimension g and channel h.

Modeling using a process of transmission and reception through a MIMO health channel subsystem, accounts for nonlinear impairments and non-Gaussian noise impacting the transmitted signals. Importantly, it enables calculation of the health capacity of a training dataset using techniques known to calculate channel capacity. By modeling the process this way, the system captures real-world complexities, allowing accurate health capacity calculations and maintaining coherence throughout the system.

As explained above, measured signal representation 1G-23 is a representation of measured signals from sensors for each dimension g and channel h, denoted as

$${s}_{R x}^{k , h , g} ( t ) .$$

In some embodiments, as is discussed below, this represents the point where:
training signals from training dataset 106;
testing signals from training dataset 106; and
measured signals from sensors 103 connected to patients;
   are used so as to generate outputs for channel h and dimension g.

In step 1I-15: the output for channel h and dimension g is extracted. To extract the output for channel h and dimension g denoted as

$${y}_{n , h}^{k , g} ,$$

the measured signals are multiplied by the representative signal for channel h and dimension g denoted as

$${φ}_{g}^{h} ( t - n T )$$

where T is the signal period, and nT≤t≤(n+1)T. The output from this operation is then integrated over the signal period T as shown below:

$$\begin{matrix}{y}_{n , h}^{k , g} = {max}_{τ} \{ \frac{{\int }_{0}^{T} {s}_{Rx}^{k , h , g} ( t ) {φ}_{h}^{g} ( t - τ - nT ) dt}{\sqrt{{\int }_{0}^{T} {s}_{Rx}^{k , h , g} ( t ) dt} \sqrt{{\int }_{0}^{T} {φ}_{h}^{g} ( t - nT ) dt}} , \\ g ∈ \{ 1 , 2 , … , G \}\end{matrix} \qquad ( Equation 12 )$$

The process ensures that the demodulated signal for each channel h and dimension g is within the expected range for accurate detection and classification. This correlator-based de-modulation technique is employed for the received signal analysis, allowing for precise extraction of the current health situation of d being monitored.

Referring to FIG. 1G: The outputs from the ADCs 1G-11-1 to 1G-11-G are then fed to corresponding multipliers 1G-13-1 to 1G-13-G, where each of these outputs are multiplied by the modulating signal associated with the multiplier. The modulating signal associated with the multiplier is the representative signal for that channel and dimension. For example, the output from ADC 1G-11-1 is multiplied by modulating signal 1G-15-1 which is the representative signal for channel 1 and dimension 1, denoted as

$${φ}_{1}^{1} ( t - n T ) .$$

The multiplication takes place in multiplier 1G-13-1.

The output signal from each of the multiplier 1G-13-1 to 1G-13-G is then passed through a corresponding one of the correlators 1G-17-1 to 1G-17-G to produce un output

$${y}_{n , 1}^{k , g} .$$

For example, the output signal from multiplier 1G-13-1 is processed in correlator 1G-17-1 to produce the output

$${y}_{n , 1}^{k , 1} .$$

Consequently, the demodulated signal for each channel h can be represented as

$${Y}_{n , h}^{k} = ⌈ {y}_{n , h}^{k , 1} , {y}_{n , h}^{k , 2} , … , {y}_{n , h}^{k , G} ] ,$$

and the aggregated signal for all channels as

$${Y}_{n}^{k} = [ {Y}_{n , 1}^{k} , {Y}_{n , 2}^{k} , … , {Y}_{n , H}^{k ,} ] .$$

Referring to FIG. 1G, the output 1G-19 from correlation receiver 123-1 is

$${Y}_{n , 1}^{k} = [ {y}_{n , 1}^{k , 1} , {y}_{n , 1}^{k , 2} , … , {y}_{n , 1}^{k , G} ] .$$

A detailed embodiment of healthcare feedback subsystem 155 is shown in FIG. 1H. The components and operation of the components are described further below.

Each output from the plurality of correlation receivers 123 is communicatively coupled to natural intelligence processing subsystem 131. Then, in step 1I-15 of FIG. 1J: Output signals such as output 1G-19 are transmitted from the plurality of correlation receivers 123 to natural intelligence processing subsystem 131. These output signals comprise the output symbols

$${Y}_{n}^{k} .$$

In step 1I-23 of FIG. 1J, NI processing subsystem 131 receives the outputs from the plurality of correlation receivers 123 and performs the necessary operations to carry out its role as the central cognitive brain or cognitive processor in intelligent healthcare transponder system 100. Then natural intelligence processing subsystem 131 processes the output

$${Y}_{n}^{k}$$

from the plurality of correlation receivers 123 to extract statistical data, such as posterior, evidence, and predictive outcome to identify the transmitted health situation based on the received constellation.

As explained above, training and testing signals from training dataset 106; and measured signals from sensors connected to patients are used to generate the output

$${Y}_{n}^{k} .$$

are three phases where output

$${Y}_{n}^{k}$$

is generated, as is now discussed. These are:
Training phase,
Testing phase, and
Client diagnosis phase.

Training phase: In the training phase, the entire training dataset 106 is utilized to generate the output

$${Y}_{n}^{k} .$$

These output symbols are then used to extract relevant distributions to calculate the health capacity and other associated parameters. The processes to perform these operations are discussed below.

Testing phase: The aim of this phase is to ensure that the intelligent healthcare transponder system 100 meets Diagnosis Error (DE) and computational complexity thresholds as set by the client or healthcare authorities. The testing process is executed using two operations: database splitting and cross-validation. Insertion as measured signal representation 1G-23

In the database splitting operation: A portion of the training dataset is set aside for testing, with the remaining portion used for model training. For example, in some embodiments, 80% of the data is allocated for training, while 20% is reserved for testing. Representative signals are selected from the remaining portion to generate the representative input symbols

$${\hat{X}}_{n}^{k , h}$$

as discussed previously. The output

$${Y}_{n}^{k}$$

is generated as discussed below. Then, using processes that are discussed below, a posterior is extracted from the training data. Using processes discussed below, the rate Rₖ and complexity threshold are adjusted to meet the DE threshold. If thresholds cannot be met for any 2≤R=Rₖ≤R_{ref}, requests are sent to the client or healthcare provider to update the thresholds or modify the database. This process continues until the thresholds is met, at which point R=Rₖ is set as the rate, and the system proceeds with cross-validation.

For the cross-validation operation: a “leave u % out” cross-validation is performed. The dataset is divided into

$$( \frac{100}{u} )$$

portions, and

$$( \frac{100}{u} )$$

iterations of training and testing are conducted. In each iteration, one portion is left out for testing, while the remaining portions are used for training. For example, when u=20%, the dataset is split into 5 portions, and 5 iterations of training and testing are performed. This approach minimizes the risk of overfitting. The posterior is extracted at each step, and representative signals are selected from the training portion. The average DE is computed to evaluate if the system meets the threshold for R=Rₖ. If the threshold is not met for any 2≤R=Rₖ≤R_{ref}, updates to the thresholds or the database are requested. This procedure continues until the specified thresholds are met.

In the client diagnosis phase, the intelligent healthcare transponder 100 diagnoses the client's health condition using the signals

$${s}_{R x}^{k , h , g} ( t )$$

received from the sensors, such as, for example, the measured signal from sensors 103 shown in FIG. 1A. Using processes that are discussed below, Rₖ which corresponds to the number of health situations is determined, and the appropriate channels h are selected for these measured signals to be used. The measured signals are multiplied by the representative signal

$${φ}_{h}^{g} ( t - n T )$$

at, for example multipliers 1G-13-1 to 1G-13-G of FIG. 1G, as explained above, and the correlation as defined in Equation 12 is calculated. The received vector

$${Y}_{n , h}^{k} = ⌈ {y}_{n , h}^{k , 1} , {y}_{n , h}^{k , 1} , ⋯ , {y}_{n , h}^{k , G} ] ,$$

is computed. Then, the posterior extracted from the training phase above is applied using equations and processes which are described below to determine the health situation. At that point, R=Rₖ is set as the number of health situations is determined, and the system proceeds with the diagnosis. The steady state is turned on, and the estimated DE is continuously monitored. If the estimated DE exceeds the threshold during steady-state operation, the steady state is turned off and attempt to find a new Rₖ that provides a DE below the threshold. If no such Rₖ can be found, the system will request the client or healthcare authorities to update the policy or thresholds. This process ensures the system maintains reliable performance while adapting to real-time conditions.

If the database is updated, the entire process from training to testing and client diagnosis will be repeated.

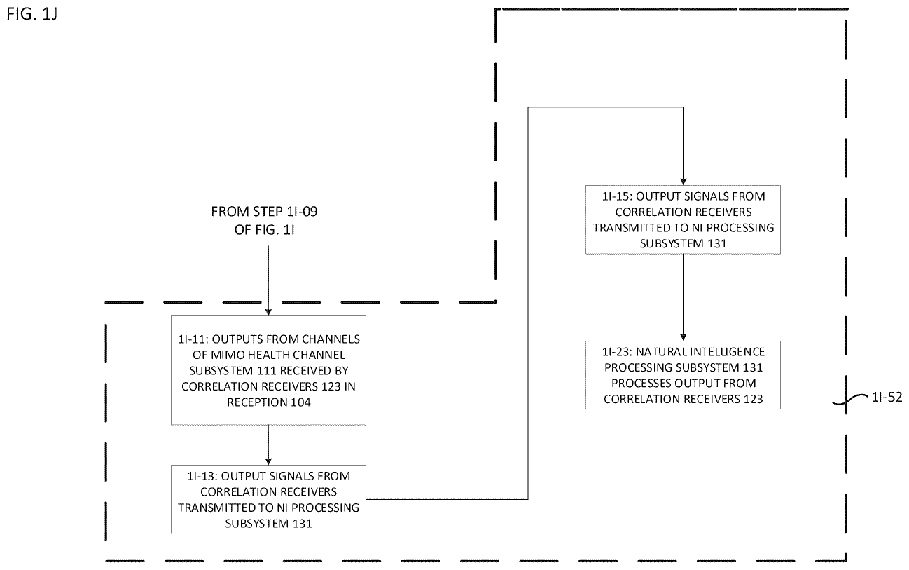

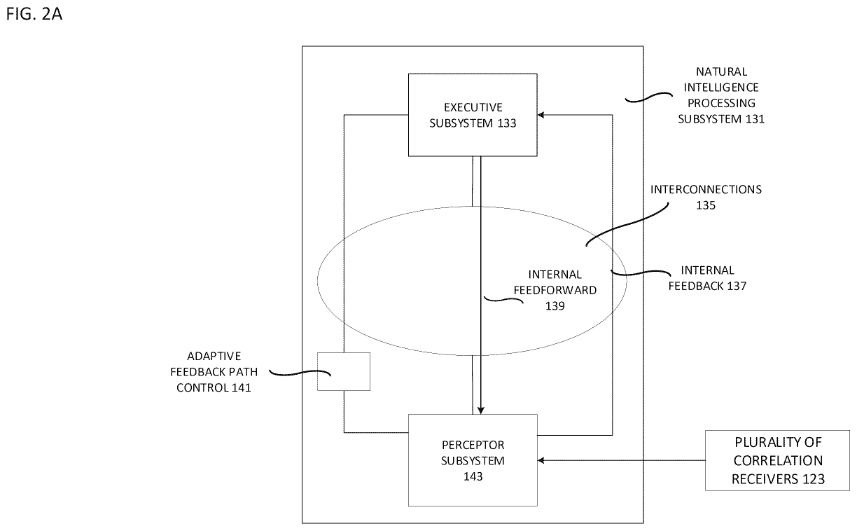

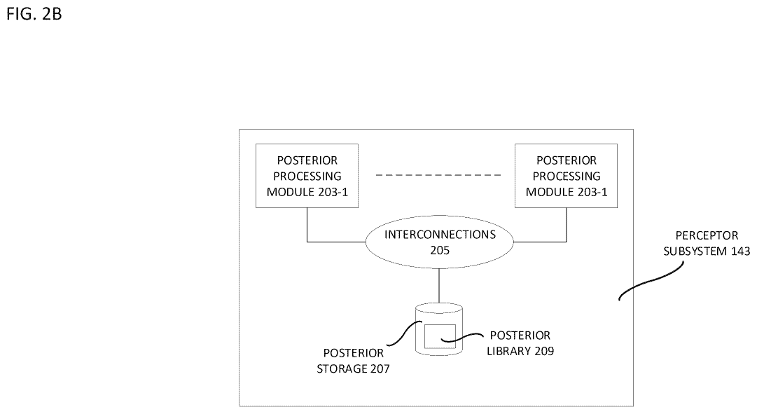

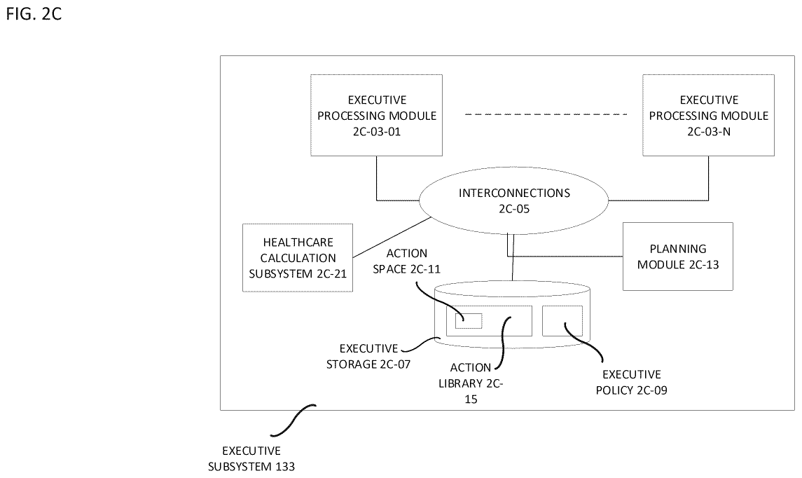

FIG. 2A shows a detailed embodiment of NI processing subsystem 131. In FIG. 2A, perceptor subsystem 143 is communicatively coupled to executive subsystem 133 via interconnections 135.

Channels are set up between perceptor subsystem 143 and executive subsystem 133 via interconnections 135. Examples of these channels are:
Internal feedforward channel 139 is set up to direct internal feedforward signals from executive subsystem 133 to perceptor subsystem 143; and
Internal feedback channel 143 is set up to direct internal feedback signals from perceptor subsystem 143 to executive subsystem 133.

The adaptive feedback path control module 141 is communicatively coupled to both perceptor subsystem 143 and executive subsystem 143 using, for example, an adaptive feedback path channel set up via interconnections 135. The adaptive feedback path control module 141 performs the role of dynamically adjusting the system's behavior based on real-time evaluations, so as to enable maintenance of system accuracy by providing real-time updates on diagnostic error (DE) and other performance metrics. The adaptive feedback path control module 141 ensures that the system remains in a steady state as long as the DE is within acceptable limits. If the DE exceeds the threshold, the adaptive feedback path control module 141 triggers adaptive actions to bring the system back to optimal performance.

In some embodiments, adaptive feedback path control module 141 is implemented in hardware. In other embodiments, adaptive feedback path control module 141 is implemented in software. In yet other embodiments, adaptive feedback path control module 141 is implemented using a combination of software and hardware.

FIG. 2B shows a detailed embodiment of perceptor subsystem 143. In FIG. 2B, perceptor subsystem 143 comprises one or more posterior processing modules 203-1 to 203-N. Posterior processing modules 203-1 to 203-N can be implemented in a variety of ways. In some embodiments, posterior processing modules 203-1 to 203-N are implemented in hardware. In other embodiments, posterior processing modules 203-1 to 203-N are implemented in software. In yet other embodiments, posterior processing modules 203-1 to 203-N are implemented in a combination of hardware and software. In some embodiments, posterior processing modules 203-1 to 203-N comprises a plurality of components.

These one or more posterior processing modules 203-1 to 203-N are communicatively coupled to posterior storage 207 via perceptor subsystem interconnections 205. Perceptor subsystem interconnections 205 are implemented using appropriate communication technologies known to those of ordinary skill in the art.

Posterior storage 207 stores posterior library 209. In some embodiments, posterior storage 207 comprises a database, which is implemented using database techniques known to those of ordinary skill in the art. Data stored in the database is indexed, using techniques known to those of ordinary skill in the art. In some embodiments, the posterior storage 207 is made searchable using techniques known to those of ordinary skill in the art. For example, the posterior storage 207 is implemented as a database.

Posterior library 209 stores, for example:
a plurality of posterior models,
a plurality of prior models,
a plurality of evidence models, and
a plurality of predictive outcome models.

Posterior models are statistical or probabilistic models which are used to predict transmitted symbols for channel h given the received symbols at channel h, thereby offering a way to characterize intelligent healthcare transponder system 100 behavior. These are denoted mathematically as

$$P ( {\hat{X}}_{n}^{k , h} \vert {\hat{Y}}_{n}^{k , h , m} ) .$$

### Referring to Equation 3:

$${\hat{X}}_{n}^{k , h} ∈ \{ {X}_{h 1} , {X}_{h 2} , … , {X}_{h D} \}$$

one of skill in the art sees that each element of the representative input symbols

$${\hat{X}}_{n}^{k , h}$$

corresponds to a class d drawn from the set {1, 2, . . . , D}.

Prior models are statistical models which capture the probability distributions of the transmitted symbols for each channel h. These are denoted mathematically as

$$P ( {\hat{X}}_{n}^{k , h} ) .$$

Evidence models are statistical models which capture the probability distributions of the received symbols for each channel h. These are denoted mathematically as

$$P ( {\hat{Y}}_{n}^{k , h , m} ) .$$

Predictive outcome models are statistical or probabilistic models which are used to predict received symbols for channel h given transmitted symbols for channel h. These are denoted mathematically as

$$P ( {\hat{Y}}_{n}^{k , h , m} \vert {\hat{X}}_{n}^{k , h} ) .$$

In some embodiments, these models are indexed using indexing parameters such as
dataset name,
date of dataset update, and
version number
These indexing parameters enable posterior library 209 to be searchable. By storing historical data, the posterior library 209 provides the system with the flexibility to respond to new or unexpected conditions and changes.

FIG. 2C shows a detailed embodiment of executive subsystem 133. In FIG. 2C, executive subsystem 133 comprises planning module 2C-13. Planning module 2C-13 performs planning tasks within executive subsystem 133. For example, planning module 2C-13 identifies and extracts a series of prospective actions from the action library 2C-15, which will be explained further below.

Additionally, the planning module 2C-13 is responsible for updating the type of actions to be taken. Planning module 2C-13 performs an update process through both internal feedback channel 137 and internal feedforward channel 139, forming a shunt cycle. This cycle allows for a dynamic adjustment of the intelligent healthcare transponder system 100 parameters in real-time, enabling the NI processing subsystem 131 to adapt to new information or changes in the environment swiftly. For example, planning module 2C-13 sends internal commands to the perceptor subsystem 133 via internal feedforward channel 139 to, for example, modify the precision factor or focus level used by one or more posterior processing modules 203-1 to 203-N. Planning module 2C-13 sends requests to one or more posterior processing modules 203-1 to 203-N to retrieve data such as posterior models from posterior library 209 or discretized data vectors. Retrieved data is sent from one or more posterior processing modules 203-1 to 203-N to planning module 2C-13 via internal feedback channel 137, for use in virtual environmental prediction, as is discussed further below. Planning module 2C-13 also performs other processing tasks either on its own or in conjunction with one or more executive processing modules 2C-03-01 to 2C-03-N as needed.

In some embodiments, planning module 2C-13 is implemented in hardware. In other embodiments, planning module 2C-13 is implemented in software. In yet other embodiments, posterior planning module 2C-13 is implemented in a combination of hardware and software. In some embodiments, planning module 2C-13 comprises a plurality of components interconnected together.

The executive subsystem 133 comprises one or more executive processing modules 2C-03-01 to 2C-03-N. In some embodiments, one or more executive processing modules 2C-03-01 to 2C-03-N is implemented in hardware. In other embodiments, one or more executive processing modules 2C-03-01 to 2C-03-N is implemented in software. In yet other embodiments, one or more executive processing modules 2C-03-01 to 2C-03-N is implemented in a combination of hardware and software. In some embodiments, one or more executive processing modules 2C-03-01 to 2C-03-N comprises a plurality of components interconnected together.

In some embodiments, one or more executive processing modules 2C-03-01 to 2C-03-N works to perform processing tasks in executive subsystem 133 which are not performed by planning module 2C-13. In some embodiments, the one or more executive processing modules work together with the planning module 2C-13 to perform the planning tasks described above.

The executive subsystem 133 comprises executive storage 2C-07. Executive storage 2C-07 is implemented using storage techniques known to those of ordinary skill in the art. In some embodiments, executive storage 2C-07 comprises a database implemented using techniques known to those of ordinary skill in the art. Executive storage 2C-07 stores action space 2C-11, action library 2C-15 and executive policy 2C-09. In some embodiments, data stored in executive storage 2C-07 is indexed, using techniques known to those of ordinary skill in the art. In some embodiments, executive storage 2C-07 is searchable.

Action library 2C-15 comprises action space 2C-11, which in turn comprises the set of all possible actions available to take in response to different conditions or scenarios. In some embodiments, the set of all possible actions available comprises pre-adaptive actions, which are predetermined actions designed to be effective before the system has had a chance to learn or adapt from experience. In some embodiments, the actions in action space 2C-11 are indexed. Action space 2C-11 further comprise environmental actions and internal commands. Environmental actions and internal commands will be further explained below, along with examples.

Executive policy 2C-09 outlines the objectives that the NI aims to achieve using the PAC. Executive policy 2C-09 sets the desired targets for intelligent healthcare transponder system 100. In some embodiments, these targets comprise a balance between accurate cognitive decision making and the associated computational costs of achieving that accuracy. Policies are either simple or complex based on the goals and the operational context of the NI.

To illustrate, the executive policy 2C-09 sets a goal known as the focus level accuracy threshold, which defines the accuracy objective of the NI processing subsystem 131 decision-making at a specific focus level m while staying within the desired complexity threshold. The focus level m provides an indication of context depth. In some embodiments, the focus level m is the number of received symbols prior to a received symbol, as will be explained below. The focus level accuracy threshold is also referred to as the ATₘ, and these two terms are used interchangeably below. In some embodiments, the ATₘ at different focus levels reflect the different computational complexity requirements at these levels. For example, at the focus level m=1 the ATₘ is higher, for example, 11% than at base focus level m=0, where it is set at 7% to recognize that the cost of computational complexity due to the more detailed modeling required at the higher level necessitates a higher accuracy to compensate. This adaptive mechanism enables the NI processing subsystem 131 to optimize performance based on the trade-offs between accuracy and computational resources, thereby making more informed decisions that align with the set policy goals. In some embodiments, the focus level accuracy threshold is set externally by clients or parties who have the necessary access credentials.

The executive subsystem 133 comprises healthcare calculation subsystem 2C-21. Healthcare calculation subsystem 2C-21 performs the health capacity calculation, which is described below. The health capacity calculation determines the maximum number of health conditions that can be reliably diagnosed based on the available dataset. In some embodiments, this calculation sets the operational limits of the system, by ensuring that diagnostics are performed within statistically reliable boundaries. Health capacity is factored into decision-making processes to maintain system accuracy and prevent overloading the diagnostic framework with more conditions than it can accurately handle.

Planning module 2C-13, executive processing modules 2C-03-01 to 2C-03-N, healthcare calculation subsystem 2C-21 and executive storage 2C-07 are coupled to each other via executive subsystem interconnections 2C-05. Executive subsystem interconnections 2C-05 are implemented using appropriate communication technologies known to those of ordinary skill in the art.

As part of step 1I-23, the one or more posterior processing modules 203-1 to 203-N in perceptor subsystem 143 extracts the posterior for channel h given by

$$P ( {\hat{X}}_{n}^{k , h} \vert {\hat{Y}}_{n}^{k , h , m} ) ;$$

predictive outcome for channel h given by

$$P ( {\hat{Y}}_{n}^{k , h , m} \vert {\hat{X}}_{n}^{k , h} ) ,$$

and evidence for channel h given by

$$P ( {\hat{Y}}_{n}^{k , h , m} )$$

from the received constellations and transmits these to the executive subsystem 133 for health capacity calculation via interconnections 135. The prior for channel h,

$$P ( {\hat{X}}_{n}^{k , h} )$$

is extracted from the representative input symbols which have been transmitted by, for example, the plurality of multi-dimensional symbol mappers 107 as previously described.

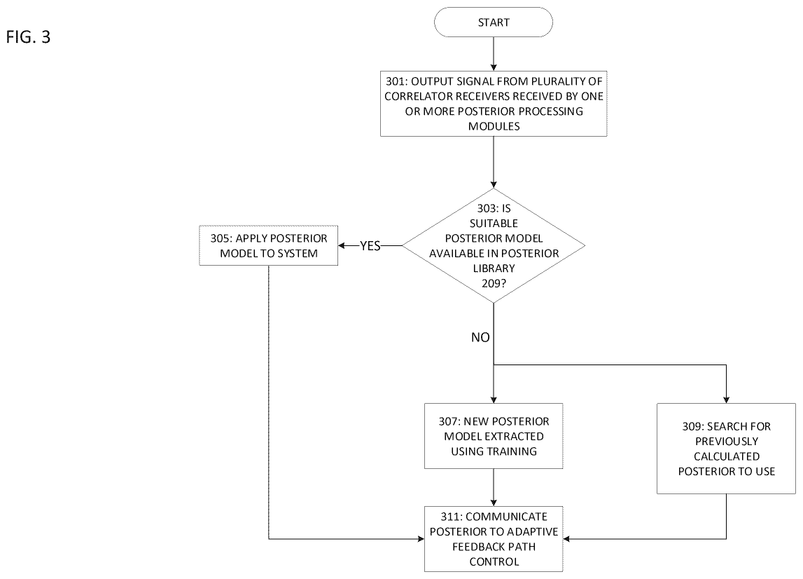

An example embodiment of a posterior processing flow is illustrated in FIG. 3. In step 301 the output signal from plurality of correlation receivers 123 comprising symbols

$${Y}_{n}^{k}$$

is received by one or more posterior processing modules 203-1 to 203-N.

Within the context of a PAC, the output signal from plurality of correlation receivers 123 comprising symbols

$${Y}_{n}^{k}$$

represents the perceptions. Based on these perceptions, appropriate actions are chosen, as explained below.

In step 303 one or more posterior processing modules 203-1 to 203-N then communicates with posterior storage 207 to search posterior library 209 with the aim of finding a suitable posterior model which captures the behavior of intelligent healthcare transponder system 100. As previously explained, in some embodiments, this comprises searching the parameters used to index the posterior model to find the closest match to the current system parameters.

When a suitable posterior model is found in step 303, this posterior model is applied to the current operational parameters of the system in step 305.

When a suitable posterior model is not found in step 303, in some embodiments, in step 307 one or more posterior processing modules 203-1 to 203-N initiates the extraction of a new posterior model to minimize the diagnostic error (DE) by extracting a fitting using model using training datasets 106.

This newly identified posterior model is then stored in the posterior library 209 for future reference and employed in subsequent decision-making processes.

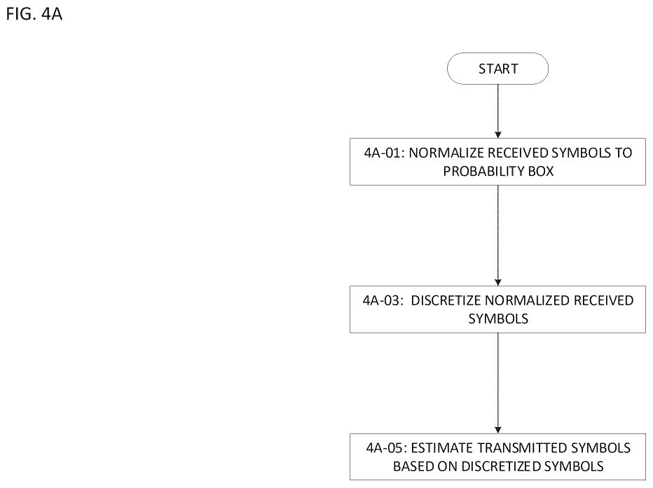

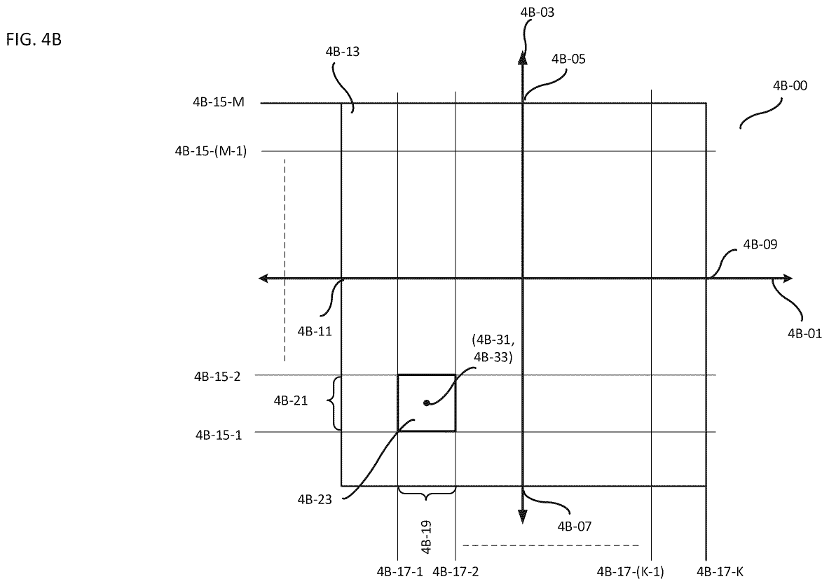

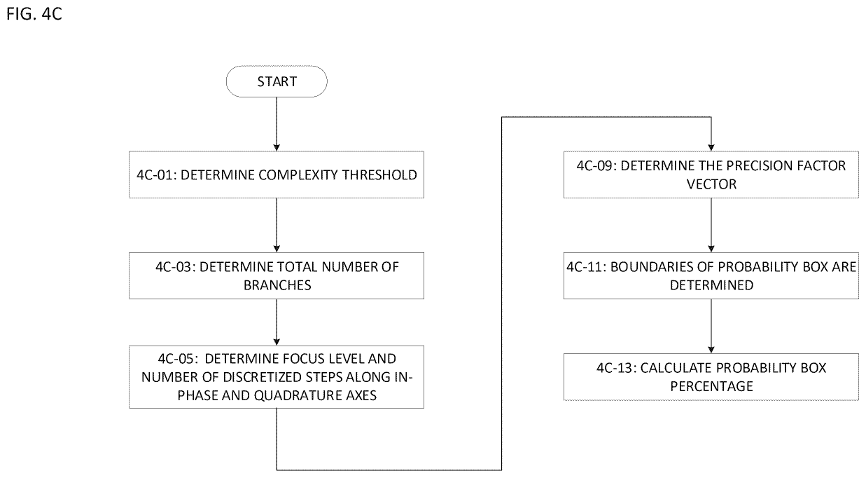

FIG. 4A shows an example embodiment of a posterior extraction processing flow using training, performed by one or more posterior processing modules 203-1 to 203-N for a focus level, m.

The output signal from the plurality of correlation receivers 123 comprises a plurality of symbols for each of the channels. This received plurality of symbols spans a broad spectrum of values. In step 4A-01, the received plurality of symbols is normalized to a probability box, to reduce the resulting complexity.

The process of normalization is described below with further reference to the diagram in FIG. 4B, for an example where the received symbols for channel

$$h {Y}_{n , h}^{k}$$

have 2 dimensions. In FIG. 4B, space 4B-00 is spanned by horizontal axis 4B-01 and vertical axis 4B-03. Probability box 4B-13 is defined in space 4B-00, wherein values that fall within the following boundaries are considered to lie within the box:
Horizontal boundaries: Minimum 4B-11 and maximum 4B-09; and
Vertical boundaries: Minimum 4B-07 and maximum 4B-05.

The probability box percentage denotes the proportion of the received plurality of symbols that fall within the probability box. In some embodiments, the axis boundaries are determined based on a probability box percentage threshold. For example, when the probability box percentage threshold is 95%, then the axis boundaries are set accordingly to obtain a probability box percentage at or above this probability box percentage threshold. In some embodiments, the probability box percentage is determined based on the estimated diagnostic error (DE) rate. Estimated diagnostic error rate is directly correlated with probability box percentage, which in turn is inversely correlated to probability box size. Then, the probability box size is increased to reduce diagnostic error rate. However larger probability box size leads to higher computational cost, as will be explained below. Then, in some embodiments, the probability box size is set so as to achieve a threshold diagnostic error rate while keeping computational cost low.

In other embodiments, the axis boundaries are set based on the available memory. This is useful when, for example, the perceptor subsystem 143 is implemented on a chip such as a field programmable gate array (FPGA) or application-specific integrated circuit (ASIC), where storage capacity is limited. A process to set the horizontal and vertical boundaries based on available memory is explained below. The relationship between storage capacity and the probability box is explained further below.

Then, for received symbols that fall within the probability box 4B-13, the normalized received symbols have the same values as the received symbol. For received symbols that fall outside probability box 4B-13, the normalized received symbols take on the horizontal and vertical values of the nearest boundaries. An example is demonstrated below. In this example, probability box 4B-13 has the following boundaries:
the horizontal minimum 4B-11 is set to −3,
the horizontal maximum 4B-09 is set to 3,
the vertical minimum 4B-07 is set to −3, and
the vertical maximum 4B-05 is set to 3.

Then, when symbol (7, −5) which falls outside the probability box is received, it is normalized to the nearest point on the boundary of the probability box, which is (3, −3).

In some embodiments, a probability box such as probability box 4B-13 is defined for each intermediate focus level i where i is between 0 and m, and PAC k. Then, the boundaries for focus level m for PAC k, are hereinafter referred to as follows:
The horizontal minimum, such as horizontal minimum 4B-11, is referred to as

$${x}_{min}^{k , h , i} ,$$

The horizontal maximum, such as horizontal maximum 4B-09, is referred to as

$${x}_{max}^{k , h , i} ,$$

The vertical minimum, such as vertical minimum 4B-07, is referred to as

$${y}_{min}^{k , h , i} ,$$

The vertical maximum, such as vertical maximum 4B-05, is referred to as

$${y}_{max}^{k , h , i} .$$

The normalized received symbol is hereinafter referred to as

$${{Y}^{′}}_{n , h}^{k , m} ,$$

where:
k is defined as the perception-action cycle (PAC) number,
h is the channel number,
n is the index of the current symbol,
m represents the focus level for perception-action cycle (PAC) number k, where m ranges from 0 to M, the maximum focus level. The focus level provides an indication of context depth. In some embodiments, the focus level m is the number of received symbols prior to received symbol n, which are used to predict transmitted symbol n.

Normalized received symbol

$${{Y}^{′}}_{n , h}^{k , m}$$

is then used for further processing.

In step 4A-03, normalized received symbol

$${{Y}^{′}}_{n , h}^{k , m}$$

is discretized. Processes and equations to set the discretization parameters are now described.

Axis discretizations are performed for the horizontal and vertical axes. In some embodiments, for each intermediate focus level i between 0 and the focus level m, dimension g, channel number h, a discretization step

$$Δ {x}_{i}^{k , h , g}$$

is defined for PAC k as follows:

$$Δ {x}_{i}^{k , h , g} = \frac{{x}_{max}^{k , h , i , g} - {x}_{min}^{k , h , i , g}}{{N}_{g}^{k , h , i}} \qquad ( Equation 13 )$$

where

$${N}_{g}^{k , h , i}$$

is the number of discretization steps for PAC k, channel number h, dimension g and intermediate focus level i.

Then, the axes are discretized based on the discretization steps. For example, in FIG. 4B, horizontal axis 4B-01 is discretized into K discretized horizontal points, wherein consecutive discretized horizontal points are separated by an horizontal discretization step. Then K is equal to

$${N}_{1}^{k , h , i} .$$

For example, consecutive discretized horizontal points 4B-17-1 and 4B-17-2 on the horizontal axis 4B-01 are separated by horizontal discretization step 4B-19.

Similarly, vertical axis 4B-07 is discretized into M discretized vertical points, wherein consecutive discretized vertical points are separated by a vertical discretization step. M is then equal to

$${N}_{2}^{k , h , i} .$$

For example, consecutive discretized vertical points 4B-17-1 and 4B-17-2 are separated by vertical discretization step 4B-21.

Based on the discretization of the horizontal and vertical axes, discretization cells are formed. For example, referring to FIG. 4B, discretization cell 4B-23 is bounded by 4B-17-1 and 4B-17-2 on the horizontal axis, and 4B-15-1 and 4B-15-2 on the vertical axis. Each cell has dimensions

$$( Δ {x}_{i}^{k , h , 1} \times Δ {x}_{i}^{k , h , 2} ) .$$

Then a precision factor is assigned for each intermediate focus level i for PAC k. In some embodiments, a horizontal precision factor is calculated based on the horizontal discretization step, and a vertical precision factor is calculated based on the vertical discretization step.

In some of the embodiments where the horizontal discretization step is the same as the vertical discretization step, the horizontal precision factor is equal to the vertical precision factor. This common precision factor is denoted as

$${PF}_{i}^{k , h} .$$

An example relationship between the precision factor, horizontal discretization step and vertical discretization step for embodiments where the horizontal discretization step is equal to the vertical discretization step is given as:

$${PF}_{i}^{k , h} = 10 Δ {x}_{i}^{k , h , 1} = 10 Δ {x}_{i}^{k , h , 2} , 0 \leq i \leq m , \qquad ( Equation 14 )$$

Then, a precision factor vector for focus level m and PAC k, PF^{k,h} which has its elements the common precision factor for each intermediate focus level i is denoted as:

$${PF}^{k , h} = [ {PF}_{0}^{k , h} , {PF}_{1}^{k , h} , … , {PF}_{i}^{k , h} , … , {PF}_{m}^{k , h} ] \qquad ( Equation 15 )$$

The number of decision tree branches

$${F}_{i}^{k , h}$$

at each intermediate focus level i is computed as the product of

$${N}_{g}^{k , h , i}$$

for all the dimensions:

$${F}_{i}^{k , h} = {\prod }_{g = 1}^{G} {N}_{g}^{k , h , i} \qquad ( Equation 16 )$$

Using Equations 1 and 2,

$${N}_{g}^{k , h , i}$$

can be computed based on the precision factor as shown below:

$${N}_{g}^{k , h , i} = \frac{10 ( {x}_{max}^{k , h , i , g} - {x}_{min}^{k , h , i , g} )}{{PF}_{i}^{k , h}} \qquad ( Equation 17 )$$

Therefore, for a probability box, a lower precision factor leads to a higher number of discretization steps, which then leads to a higher number of decision tree branches at each intermediate focus level. This has an impact on computational cost as will be seen below.

The total number of branches for the focus level m, hereinafter referred to as

$${F}_{m}^{total , k} ,$$

is calculated by the product of the branches at each level:

$${F}_{m}^{total , k} = {\sum }_{h = 1}^{H} {\prod }_{i = 0}^{m} {F}_{i}^{k , h} \qquad ( Equation 18 )$$

One of ordinary skill in the art would recognize that

$${F}_{m}^{total , k}$$

grows exponentially with the focus level m. Since the memory needed for storage is related to

$${F}_{m}^{total , k} ,$$

one of ordinary skill in the art would also recognize that the memory needed for storage also grows exponentially with focus level m.

One of ordinary skill in the art would also recognize from the above that a lower precision factor leads to a higher

$${F}_{m}^{total , k}$$

which leads to higher memory requirements. However, a lower precision factor leads to lower diagnostic error rate. Therefore, there is a trade-off between lowering DE and memory requirements.

In some embodiments,

$${F}_{m}^{total , k}$$

is constrained by a predefined complexity threshold based on the available memory capacity, that is:

$${F}_{m}^{total , k} \leq Complexity threshold \qquad ( Equation 19 )$$

One of ordinary skill in the art would recognize from the equations above that there are a number of approaches to set each of the measures denoted above, and tradeoffs with each set of parameters. An example embodiment of a process to determine discretization parameters starting from a known complexity threshold is shown in FIG. 4C.

In step 4C-01, the complexity threshold is determined. In some embodiments, this is performed based on the available memory. The available memory is, for example, memory available on a hard disk or for storage in a random-access memory (RAM).

In step 4C-03, the total number of branches is determined based on the complexity threshold, for example, the equation described above.

In step 4C-05, the focus level m is set, and for each intermediate focus level i between 0 and m,

$${N}_{g}^{k , h , i}$$

are determined. In some embodiments,

$${N}_{g}^{k , h , i}$$

is set equal for all intermediate focus levels. In some embodiments, since the total number of branches grows exponentially with focus level m, then focus level m is set based on the natural logarithm of the total number of branches determined in step 4C-03.

In step 4C-09, the elements of the precision factor vector

$$P {F}_{i}^{k , h}$$

are determined.

In step 4C-11 based on the elements of precision factor vector determined in step 4C-09 and the

$${N}_{g}^{k , h , i}$$

determined in step 4C-07: the discretization steps

$$Δ {x}_{i}^{k , h , g}$$

are determined, then the boundaries of the probability box are determined for each intermediate focus level i from 0 to m.

In step 4C-13 the probability box percentage is calculated. In some embodiments, this is compared to a probability box percentage threshold to determine whether the calculated probability box percentage is acceptable.

An example of the operation of FIG. 4C for a particular embodiment is now detailed, for the MIT database, which has L=G=2 and D=H=18. In step 4C-01, a complexity threshold of 10⁷ memory elements is set based on, for example, available memory.

Then, in step 4C-03, the total number of branches is:

$${F}_{m}^{total , k} \leq {10}^{7} \qquad ( Equation 20 )$$

For step 4C-05: for this embodiment

$${N}_{1}^{k , h , i}$$

is set equal to

$${N}_{2}^{k , h , i} = N .$$

### Then:

$${F}_{m}^{total , k} = {\sum }_{h = 1}^{18} {\prod }_{i = 0}^{m} {F}_{i}^{k , h} \qquad ( Equation 21 )$$

$${F}_{m}^{total , k} = 18 {N}^{2 ( m + 1 )}$$

$$m \leq \frac{ln ( 1 {0}^{7} ) - ln ( 18 )}{2 ln ( N )} - 1$$

$$m \leq \frac{13.23}{2 ln ( N )} - 1$$

Since the combination of m=1 and N=25 fulfils this requirement, in this embodiment, m is set to 1 and N is set to 25. Then, 18N^{2(m+1)}=18(25)²⁽²⁾=7,031,250 memory elements are needed, which is less than 10⁷.

In step 4C-09, PF^{k,m} is set to

$$[ {PF}_{0}^{k} , {PF}_{1}^{k} ] = [ 5 , 5 ] .$$

In step 4C-11, the discretization steps

$$Δ {x}_{i}^{k , h , 1} and Δ {x}_{i}^{k , h , 2}$$

are calculated as 0.5 using, for example, Equation 2. Then, since N=42, from Equation 1,

$$( {x}_{max}^{k , h , i , 1} - {x}_{min}^{kh , i , 1} ) = 25 \times 0.5 = 12.5 .$$

### Similarly

$$( {x}_{max}^{k , h , i , 2} - {x}_{min}^{k , h , i , 2} ) = 12 \text{.5} .$$

Based on this and centering the PB on the origin,

$$\vert {x}_{max}^{k , i} \vert = \vert {x}_{min}^{k , i} \vert = \vert {y}_{max}^{k , i} \vert = \vert {y}_{min}^{k , i} \vert = 6.25 , for i = 0 and 1.$$

Then in step 4C-13, the PB percentage is calculated for the PB defined above, where:

$${x}_{min}^{k , h , i , 1} = - 6.25$$

$${x}_{max}^{k , h , i , 1} = 6.25 ,$$

$${x}_{min}^{k , h , i , 2} = - 6 \text{.25} , and$$

$${x}_{max}^{k , h , i , 2} = 6.25 .$$

One of ordinary skill in the art would know that the example embodiment demonstrated in FIG. 4C is one example embodiment, and many embodiments are possible. In another example embodiment:
The probability box percentage threshold is set based on, for example, a DE threshold as explained previously;
The probability box boundaries are defined so as to achieve a probability box percentage at or above the probability box percentage threshold, as explained previously;
PF^{k,h} and the discretization steps are calculated;
m and N are set based on the calculation of PF^{k,h}
the total number of branches

$${F}_{m}^{total , k}$$

and memory requirement is computed and compared to the complexity threshold, and
The DE is estimated and compared to the DE threshold to ensure that the DE threshold requirement is met.

The process of discretization is now explained. Each normalized received symbol

$${\overline{Y}}_{n , h}^{k , m}$$

is converted to a discretized data symbol element for each intermediate focus level i between 0 and m based on the location of the discretized cell it falls into in space 4B-00. This discretized data symbol element is hereinafter referred to as

$${\hat{Y}}_{n - i}^{k , h} ( {PF}_{i}^{k , h} ) .$$

For example, referring to FIG. 4B, when a normalized received symbol falls into cell 4B-23, it is converted to a discretized data symbol element comprising horizontal and vertical co-ordinates assigned to cell 4B-23. In some embodiments, the mid points between the boundaries are assigned to the cell. Referring to cell 4B-23 of FIG. 4B the midpoint 4B-31 between 4B-17-1 and 4B-17-2; and the midpoint 4B-33 between 4B-15-1 and 4B-15-2 are assigned to cell 4B-23. Then any normalized received symbol which falls into the cell is converted to a discretized data symbol element comprising these assigned co-ordinates (4B-31, 4B-33).

A discretized data vector

$${\hat{Y}}_{n}^{k , h , m}$$

is then formed, comprising the discretized data symbol elements for all the intermediate focus levels between 0 and m, defined as:

$${\hat{Y}}_{n}^{k , m , h} = [ {\hat{Y}}_{n - m}^{k ,} ( {PF}_{m}^{k , h} ) , {\hat{Y}}_{n - m + 1}^{k , h} ( {PF}_{m - 1}^{k , h} ) , … , {\hat{Y}}_{n - i}^{k - h} ( {PF}_{i}^{k , h} ) , … , {\hat{Y}}_{n}^{k , h} ( {PF}_{0}^{k , h} ) ]$$

The discretized data vector

$${\hat{Y}}_{n}^{k , m , h}$$

and the precision factor vector PF^{k,h,m} are then used for further processing in step 4A-05.

In step 4A-05, the one or more posterior processing modules 203-1 to 203-N creates an estimate

$${\hat{X}}_{n , h}^{k}$$

of the transmitted symbol

$${X}_{n , h}^{k}$$

based on the discretized data vector

$${\hat{Y}}_{n}^{k , m , h} .$$

The probability

$$P ( {X}_{n , h}^{k} \vert {Y}_{n , h}^{k , m} )$$

is approximated using the Monte Carlo method by considering the probability of

$${\hat{X}}_{n , h}^{k}$$

for a given discretized data vector

$${\hat{Y}}_{n}^{k , m , h} ,$$

denoted as

$$P ( {\hat{X}}_{n , h}^{k} \vert {\hat{Y}}_{n}^{k , m , h} ) .$$

In some embodiments, a Bayesian equation is utilized to extract the posterior as follows:

$$\begin{matrix}P ( {X}_{n , h}^{k} \vert {Y}_{n , h}^{k , m} ) = \frac{P ( {Y}_{n , h}^{k , m} \vert {X}_{n , h}^{k} ) P ( {X}_{n , h}^{k} )}{P ( {Y}_{n , h}^{k , m} )} \\ ≅ \frac{P ( {\hat{Y}}_{n}^{k , m , h} \vert {\hat{X}}_{n , h}^{k} ) P ( {\hat{X}}_{n , h}^{k} )}{P ( {\hat{Y}}_{n , h}^{k , m} )}\end{matrix} \qquad ( Equation 22 )$$

For each cell, computing the probabilities P(.) requires one real division. An estimate of the computational cost is provided as follows: When the symbols

$${X}_{n , h}^{k}$$

are equally probable, the computational cost for evaluating

$$P ( {X}_{n , h}^{k} \vert {Y}_{n}^{k , m , h} )$$

is 3 real divisions per cell. Therefore, the total computational cost for extracting the posterior is

$$3 \times D \times {F}_{m}^{total , h , k}$$

real divisions, where D is the number of classes in the training dataset, and

$${F}_{m}^{total , h , k}$$

is the number of cells.

Thus, the total posterior for all MIMO channels can be expressed as:

$$P ( {X}_{n}^{k} \vert {Y}_{n}^{k , m} ) = \{ P ( {X}_{n , 1}^{k} \vert {Y}_{n , 1}^{k , m} ) , P ( {X}_{n , 2}^{k} \vert {Y}_{n , 2}^{k , m} ) , … , P ( {X}_{n , H}^{k} \vert {Y}_{n , H}^{k , m} \}$$

Following this: one or more of the posterior processing modules 203-1 to 203-N uses the posterior

$$P ( {\hat{X}}_{n}^{k} \vert {\hat{Y}}_{n}^{k , m} )$$

to select the X_{d} that has the maximum probability for each discretized cell from the Rₖ, as shown in equation 23 (steady state or testing phase using portion of database):

$$\overline{X} = {arg max}_{d = 1 , 2 , … , {R}_{k}} \{ {\sum }_{h = 1}^{H} P ( {\hat{X}}_{n}^{k , h} = {X}_{d} \vert {\hat{Y}}_{n}^{k , h , m} ) \} , \qquad ( Equation 23 )$$

$$\overline{X} ∈ \{ 1 , 2 , … , {R}_{k} \}$$

Here, Rₖ represents the number of coordinates considered per channel number h in PAC number k, where Rₖ is set by executive subsystem 133 based on the health capacity calculation which is described below. For example, when Rₖ=5, then the 5 most prevalent health situations or conditions are contemplated. Since there are two sensors: there are 5 coordinates considered, wherein each co-ordinate has 2 points.

Then, X̅ in Equation 23 is the health condition for which the maximum of the summation is recorded. The maximum posterior is stored in the posterior library 209 as a matrix

$$P max ( \overline{X} , {\hat{Y}}_{n}^{k , m} ) = max \{ {\sum }_{h = 1}^{H} P ( {\hat{X}}_{n}^{k , h} = {X}_{d} \vert {\hat{Y}}_{n}^{k , h , m} ) \} ,$$

along with the corresponding X̅. During steady state operation, if the received data, after discretization and normalization, matches a specific cell

$${\hat{Y}}_{n}^{k , m} ,$$

the corresponding X̅ from the matrix

$$P max ( \overline{X} , {\hat{Y}}_{n}^{k , m} )$$

is estimated as the most likely health situation.

By saving only the maximum posterior, memory requirements are significantly reduced. For instance, if there are two sensors and 16 health situations, the memory reduction factor is 256 (D×D). Additionally, since

$$P max ( \overline{X} , {\hat{Y}}_{n}^{k , m} )$$

is evaluated during the training period, then the estimation of health situation during the steady state is expedited.

This continuous updating and refinement of the posterior library 209 allows for improvement of decision-making capabilities over time, ensuring both adaptability and precision in maintaining optimal network performance.

In other embodiments, when a model cannot be found, in step 309 the one or more posterior processing modules 203-1 to 203-N searches for a previously used posterior model, stored in posterior library 209, which yields the best DE estimation.

By comparing the estimated DE against the stored posterior models, the one or more posterior processing modules 203-1 to 203-N identifies the most accurate model or decision rules applied in the past. This process enables the perceptor to refine its predictions and adjustments for future data processing and decision-making, ultimately enhancing the overall reliability and performance of the system.

Then, the searching comprises finding the closest match to the current parameters.

In some embodiments, step 307 and 309 are performed in parallel.

In step 311, the one or more posterior processing modules 203-1 to 203-N relays the selected posterior model to the adaptive feedback path control module 141. As explained previously, the adaptive feedback path control 141 estimates DE. In the steady state, the adaptive feedback path control 141 utilizes an assurance factor derived from the relayed posterior model relayed from the perceptor subsystem 143. When the system is not in steady state, the adaptive feedback path control 141 calculates DE from received training data. In both cases, the estimated or calculated DE is relayed to executive subsystem 133.

Assurance factor is now explained. The assurance factor offers a direct measure of confidence or probability that the system has correctly identified or decided on the transmitted symbol, given the received symbol.

The average assurance factor (AF) is defined as the mean of the posterior probabilities over a discrete time interval B, expressed as:

$${AF}_{n}^{k} = \frac{{arg max}_{h = 1 , 2 , … , H} \{ {\sum }_{o = n - B}^{n} P ( {\overline{X}}_{o}^{k , h} \vert {\hat{Y}}_{o}^{k , h , m} ) \}}{B} , ( n - B ) \geq m \qquad ( Equation 24 )$$

In this equation, B represents an arbitrary discrete time interval used for calculating the assurance factor, n denotes the current time, and m indicates the focus level.

During steady-state operation, when training data is unavailable, the adaptive feedback path control module 141 estimates the DE for the kth Perceptual Adaptive Control (PAC) as the complement of the assurance factor

$${AF}_{n}^{k} .$$

The estimated DE, denoted as

$$< {DE}_{k}^{n} > ,$$

is given by:

$$< {DE}_{k}^{n} > = 1 - {AF}_{n}^{k} \qquad ( Equation 25 )$$

The assurance factor (AF) is defined as (1−probability of error), thus providing a direct measure of confidence or the probability that the system has correctly identified or decided on the transmitted symbol given the received symbol. For simplicity, this is represented as

$$〈 {DE}_{k}^{n} 〉 ,$$

which is inversely correlated with the assurance factor, thereby offering a quantifiable measure of reliability. This equation enables the estimation of DE based on the assurance factor, which serves as a measure of confidence in the system's accuracy in identifying or deciding on the transmitted symbol, given the received symbol.

Furthermore, the one or more posterior processing modules 203-1 to 203-N can utilize this estimated DE to evaluate which previously calculated posterior probabilities, stored in the library, yielded the most accurate DE estimation. By comparing the estimated DE against the stored posteriors, the system can identify the most accurate models or decision rules applied in past scenarios. This iterative process allows the one or more posterior processing modules 203-1 to 203-N to refine its predictions and adjustments for future data processing and decision-making, ultimately enhancing the overall reliability and performance of the system.

The healthcare calculation subsystem 2C-21 in executive subsystem 133 receives the posterior, prior, model and evidence; and performs a health capacity calculation. The health capacity calculation is based on a calculation of Shannon channel capacity for the channels of the MIMO health channel subsystem.

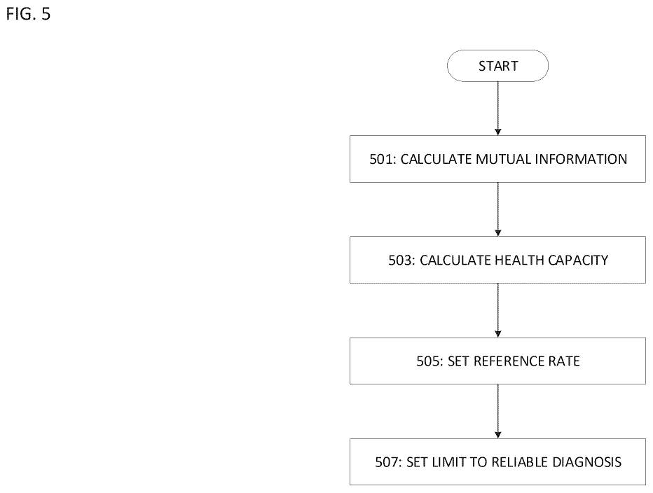

An example embodiment of the health capacity calculation is described below, with reference to FIG. 5.

In step 501: For channel number h of the MIMO health channel subsystem, mutual information is calculated using the following formula:

$${I}_{h} ( X ; Y ) ≅ {\sum }_{{\hat{Y}}_{n}^{k , h , m} ∈ {Y}_{D}} {\sum }_{{\hat{X}}_{n}^{k , h} ∈ {X}_{D}} P ( {\hat{X}}_{n}^{k , h} \vert {\hat{Y}}_{n}^{k , h , m} ) P ( {\hat{Y}}_{n}^{k , h , m} ) {log}_{2} ( \frac{P ( {\hat{Y}}_{n}^{k , h , m} \vert {\hat{X}}_{n}^{k , h} )}{P ( {\hat{X}}_{n}^{k , h} )} ) \qquad ( Equation 26 )$$

In step 503: The health capacity for channel h, referred to as

$${C}_{health}^{h}$$

below is calculated based on this mutual information. The health capacity calculation is based on whether the prior is fixed or customizable. When the prior is fixed, it cannot be modified to maximize mutual information.

Examples of customization comprise methods such as data augmentation to balance the dataset for channel number h. When the prior is fixed, the health capacity for channel h is given as:

$${C}_{health}^{h} = {I}_{h} ( X ; Y )$$

When the prior is customizable, the health capacity for channel h is given as:

$${C}_{health}^{h} = {sup}_{{P}_{X}^{h} ( x )} {I}_{h} ( X ; Y )$$

Thus, the maximum possible total health capacity for H channels of the health MIMO channel is given by:

$${C}_{health} = {\sum }_{h = 1}^{H} {C}_{health}^{h} \qquad ( Equation 27 )$$

The term health capacity and Cₕₑₐₗₜₕ are used interchangeably for the rest of this specification.

For example: for the MIT database: utilizing the prior probability

$$P ( {\hat{X}}_{n}^{k , h} )$$

from the database, and since the prior is fixed, the calculated health capacity Cₕₑₐₗₜₕ is approximately 2.399 bits per symbol.

In step 505: The calculated Cₕₑₐₗₜₕ value is then utilized by, for example, at least one of the one or more executive processing modules 2C-03-01 to 2C-03-N and planning module 2C-13 within executive subsystem 133 to set the reference rate R_{ref}=floor(2^{C}^{ₕₑₐₗₜₕ}). For example, for the MIT database, floor (2^{2.399})=floor(5.273)=5, indicating that a maximum of five (5) health situations can be accurately classified. This aligns with the findings of numerous published studies over several years, which demonstrate that accurate classification of more than five classes is not feasible with the MIT database.

In step 507: the calculated reference rate is then used by planning module 2C-13 in steady-state mode as a limit to reliable diagnosis within the intelligent healthcare transponder system 100. That is, in steady-state mode, the maximum health situations considered for diagnosis will be R_{ref}. For example, in some embodiments, the five most prevalent classes are selected by as the classes for further processing.

In some embodiments, as part of step 507: the planning module 2C-13 verifies that the current R_{ref} is sufficient relative to the required capacity for the health situation being analyzed. When the reference rate is adequate, the system proceeds with the current operations; otherwise, it recalculates thresholds or takes corrective actions.

The planning module 2C-13 attempts, through actions and by leveraging the dataset, to maintain R_{ref} while minimizing the diagnosis error. When planning module 2C-13 determines that maintaining R_{ref} is leading to higher errors, or less than R_{ref} classes should be considered to reduce or minimize diagnosis error; then as part of step 507: in some embodiments planning module 2C-13 lowers the number of health situations considered, effectively adjusting R_{ref} during Perception-Action Cycles (PACs). For example, when the MIT database is used as a training dataset 106, the planning module 2C-13 reduce the number of classes, for to four or fewer, to ensure that the intelligent health operates within acceptable diagnosis error (DE) thresholds. In some embodiments, this entails using only the four most prevalent classes or fewer. This dynamic adjustment allows the system to remain robust and accurate, even when faced with varying and complex health data conditions.

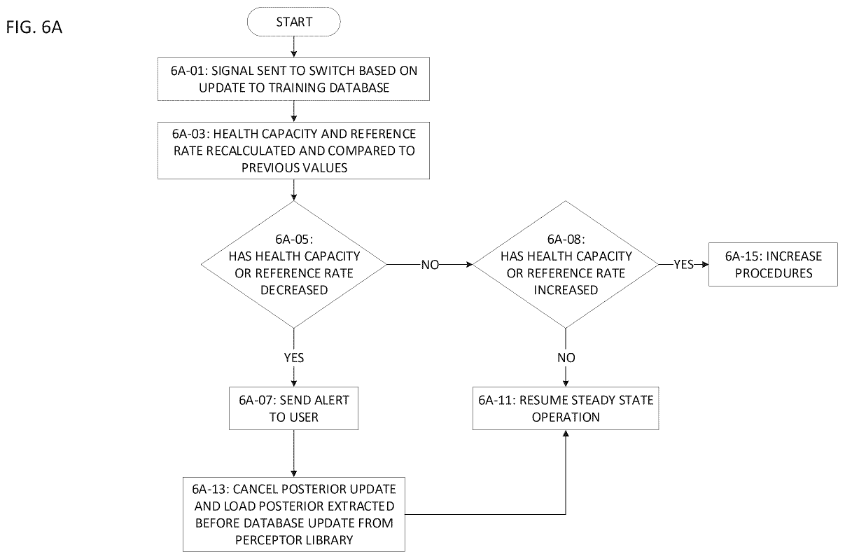

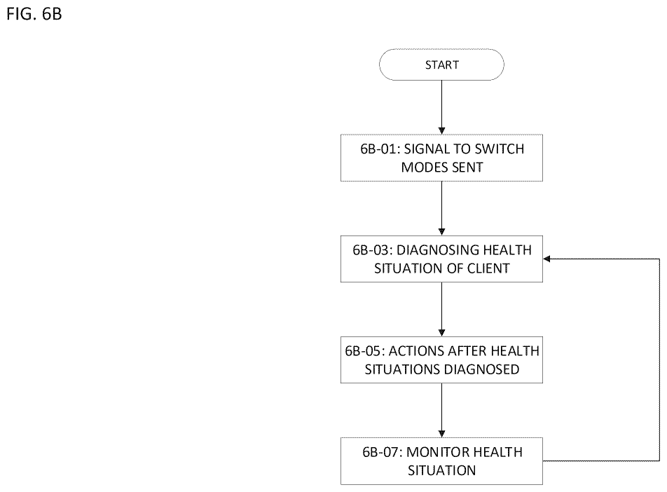

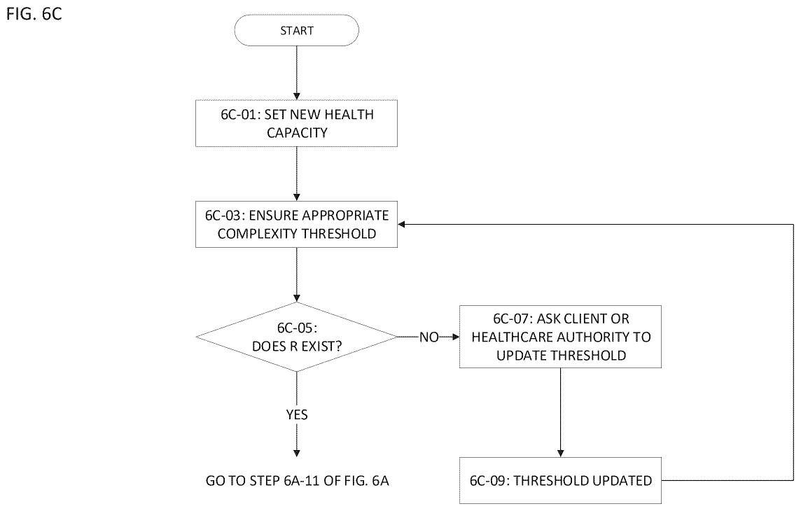

Additionally, this information provides valuable insights to the NI processing subsystem 131 in scenarios involving updates to the database. FIG. 6A shows an example embodiment of a process following a training dataset 106 update.

In step 6A-01: based on at least one of:
the executive subsystem 133 detecting that the training dataset 106 is updated, or executive subsystem 133 receiving a signal indicating that the training dataset 106 is updated;
directives sent to the executive subsystem 133 by, for example, healthcare authorities; or
initial deployment of the intelligent healthcare transponder system 100;
   The intelligent healthcare transponder system 100 switches to training mode, and the one or more executive processing modules 2C-03-01 to 2C-03-N sends an appropriate signal to healthcare feedback subsystem 155 so as to send an adjustment to the switch 105. Planning module 2C-13 sets the initial rate R=D, where D represents the maximum number of classes recorded in the training dataset 106.

In step 6A-03: using the above-described processes Cₕₑₐₗₜₕ and R_{ref} are recalculated and compared to previous values by healthcare calculation subsystem 2C-21.

In step 6A-05: the comparison of recalculated values to previously calculated values is used to determine when a decrease has occurred.

Issues such as mis-annotation or human error can lead to a decrease in Cₕₑₐₗₜₕ, contrary to the expectation that capacity should increase with updates. In step 6A-07 when a decrease in the Cₕₑₐₗₜₕ and R_{ref} is detected, an indication of mis-annotation or human error is provided by, for example, executive subsystem 131 sending a signal to healthcare feedback subsystem 155 to send an alert notification of decreased capacity to a user via healthcare feedback subsystem 155.

In step 6A-13, both executive subsystem 133 and perceptor subsystem 143 cancel any ongoing procedures. One or more posterior processing modules 203-1 to 203-N reloads the previously stored posterior data from the posterior library 209, and continues to use the pre-existing posterior data for diagnosing the client's health situation and recommending health-related actions until the issue is resolved by the client or healthcare authorities.

In step 6A-11, steady state operation is resumed within intelligent health transponder system 100. An example embodiment of a process to resume steady state operation is now described in FIG. 6B.

In step 6B-01, a signal to switch modes from training to steady state is transmitted to switch 105 from healthcare feedback subsystem 155, based on a signal sent by at least one of planning module 2C-13 and one or more executive processing modules 2C-03-01 to 2C-03-N.

In step 6B-03, a health situation of the patient is diagnosed by one or more posterior processing modules 203-1 to 203-N using Equation 23 as previously discussed.

In step 6B-05, actions as described before are undertaken.

In step 6B-07, the health situation of the patient is continually monitored. In some embodiments, this comprises reverting to step 6B-03 to diagnose the health situation of the client using Equation 23.

Conversely, when in step 6A-08 the maximum health situations R_{ref} increases following a database update or during the initial deployment of the intelligent healthcare transponder system 100, then at least one of one or more executive processing modules 2C-03-01 to 2C-03-N and planning module 2C-13 initiates a process related to the increase in R_{ref}. An example embodiment of a flow for such a process is shown in FIG. 6C.

In step 6C-01, a new health capacity is set by healthcare calculation subsystem 2C-21 using the previously described operations based on, for example, a command sent by at least one of one or more executive processing modules 2C-03-01 to 2C-03-N and planning module 2C-13.

In step 6C-03, at least one of one or more executive processing modules 2C-03-01 to 2C-03-N and planning module 2C-13 then initiates procedures to minimize the diagnosis error (DE), aiming to achieve a desired DE within acceptable complexity thresholds.

In step 6C-05, a procedure to determine whether an optimal value R such that 2≤R=Rₖ≤R_{ref} and DE^{K}≤ATₘ exists is performed by at least one of one or more executive processing modules 2C-03-01 to 2C-03-N and planning module 2C-13.

When in step 6C-05, a suitable R that satisfies the threshold conditions is identified, the intelligent healthcare transponder 100 reverts to steady-state mode as described in step 6A-11 of FIG. 6A.

When in step 6C-05, no suitable R is found that meets the required DE and complexity thresholds set by the client or healthcare authorities, in step 6C-07 at least one of one or more executive processing modules 2C-03-01 to 2C-03-N and planning module 2C-13 requests an update to these thresholds, suggesting possible values based on the minimum DE achieved for different Rₖ during the perception-action cycles, where k∈{1, 2, . . . , K}, and K is the maximum number of cycles performed by the NI processing subsystem 131. This request is sent, for example, by healthcare feedback subsystem 155 based on signals sent by at least one of one or more executive processing modules 2C-03-01 to 2C-03-N and planning module 2C-13.

Until these thresholds are updated, the NI will continue to operate using the minimum DE achieved and the corresponding R as the best option during steady-state mode, awaiting further instructions from the client or healthcare authorities.

Once these thresholds are updated, then the flow returns to step 6C-03 to ensure that the complexity threshold is appropriate.

A similar process to that detailed in FIG. 6C is carried out with regard to the database splitting operation and cross-validation operation carried out during the testing phase as discussed previously.

This information is also useful to detect potential cyber-attacks that manipulate the training data or database. In some embodiments, when a decrease in Cₕₑₐₗₜₕ and R_{ref} are detected in step 6A-05, then in step 6A-07 an alert notification is sent to a user by healthcare feedback subsystem 155 based on a signal sent by at least one of one or more executive processing modules 2C-03-01 to 2C-03-N and planning module 2C-13. In some embodiments, an entry log is checked by, for example, healthcare feedback subsystem 155 for modifications to the training dataset 106. In some embodiments, when a suspicious modification is detected, it is flagged and a further notification is sent to the user by healthcare feedback subsystem 155. In other embodiments, the alert notification contains a message advising the user to conduct a cybersecurity scan for an attack.

In yet other embodiments, the system and method described above are used to determine when potentially suspicious results are produced in, for example, a research publication. Then, the health capacity of the training dataset 106 used in the publication is evaluated by healthcare calculation subsystem 2C-21 using the systems and methods above and compared to the results in the research publication. When, for example, the number of classes stated in the research publication exceeds the healthcare capacity, an alert is sent to the user indicating a potentially suspicious result. This alert is sent by, for example, healthcare feedback subsystem 155 based on signals sent by at least one of one or more executive processing modules 2C-03-01 to 2C-03-N and planning module 2C-13.

One of ordinary skill in the art would appreciate that the above can be used to evaluate training datasets with D classes, and each of the data points in the classes drawn from L sensors, in settings other than healthcare. One of ordinary skill in the art would appreciate that the above can be used to evaluate training datasets with D classes, and each of the data points in the classes having L dimensions. Then, instead of health capacity, effective training dataset capacity is calculated.

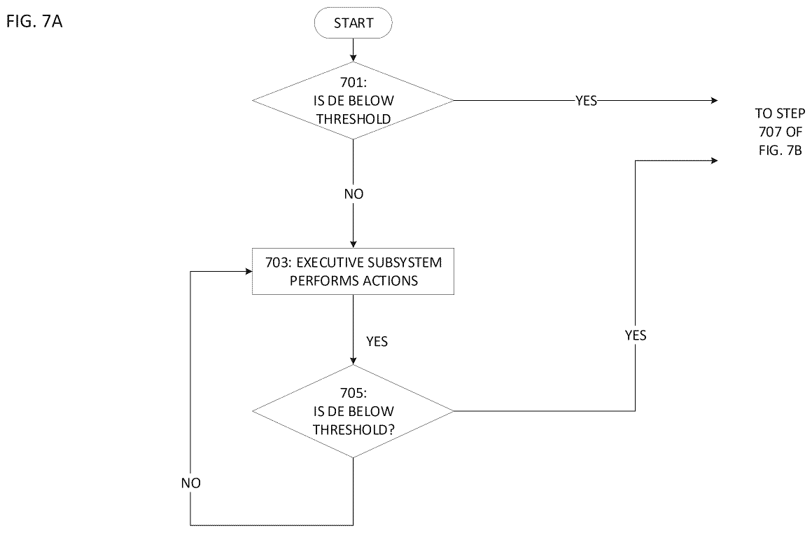

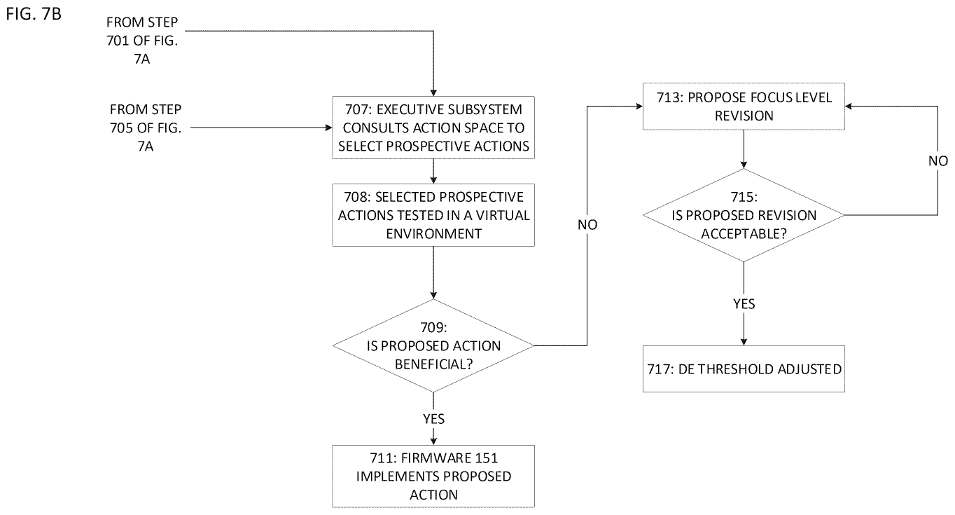

An illustrative embodiment of an executive subsystem process flow is shown in FIGS. 7A and 7B. In step 701 of FIG. 7A, the received estimated DE is compared against a predefined threshold set by executive policy 2C-09. As explained previously the threshold is set by either a client or a party having an authorized credential.

When the estimated DE is below the threshold, the planning module 2C-13 consults the action space to select a prospective action, as shown in step 707 of FIG. 7B.

When the estimated DE is above the threshold and the system is in steady state mode, in step 703 at least one of one or more executive processing modules 2C-03-01 to 2C-03-N and planning module 2C-13 disengages the intelligent healthcare transponder system 100 from steady state mode and transitions intelligent healthcare transponder system 100 into training mode.

As part of this transition, the executive subsystem 133 sends a signal to feedback subsystem 155. Then, feedback processing module 1D-07 within feedback subsystem 155 sends a signal to switch 105 in transmission 102 to perform necessary actions. Feedback processing module 1D-07 within feedback subsystem 155 is discussed in detail further below.

The executive subsystem 133 also communicates to the adaptive feedback path control module 141 that the intelligent healthcare transponder system 100 is being disengaged from steady state mode and is being transitioned into training mode.

When at least one of one or more executive processing modules 2C-03-01 to 2C-03-N and planning module 2C-13 determines that the DE is below the threshold, then planning module 2C-13 consults the action space to select a prospective action in step 507 of FIG. 5.

When at least one of one or more executive processing modules 2C-03-01 to 2C-03-N and planning module 2C-13 determines that the DE is above the threshold, the process returns to step 703, and necessary actions are performed. When there are no suitable alternative models remaining in posterior library 209 in step 505A, the planning module 2C-13 of executive subsystem 133 initiates pre-adaptive actions in step 505B. As explained previously, pre-adaptive actions are predetermined actions designed to be effective before the intelligent healthcare transponder system 100 has had a chance to learn or adapt from experience. These pre-adaptive actions comprise, for example, reducing the rate incrementally to align the DE with acceptable levels.

When there is a suitable alternative model, then the process returns to step 503, where the adaptive feedback path 141 retrieves the suitable alternative posterior model from the posterior library 209, and tests to determine whether the DE is below the threshold in step 504. When the adaptive feedback path 141 determines that the DE is below the threshold, then the executive subsystem consults the action space to select a prospective action in step 507 of FIG. 5.

Once the DE is below the threshold, in step 707 the planning module 2C-13 in executive subsystem 133 consults the action space 2C-11 in executive storage 2C-07 to select prospective actions for adjusting parameters.

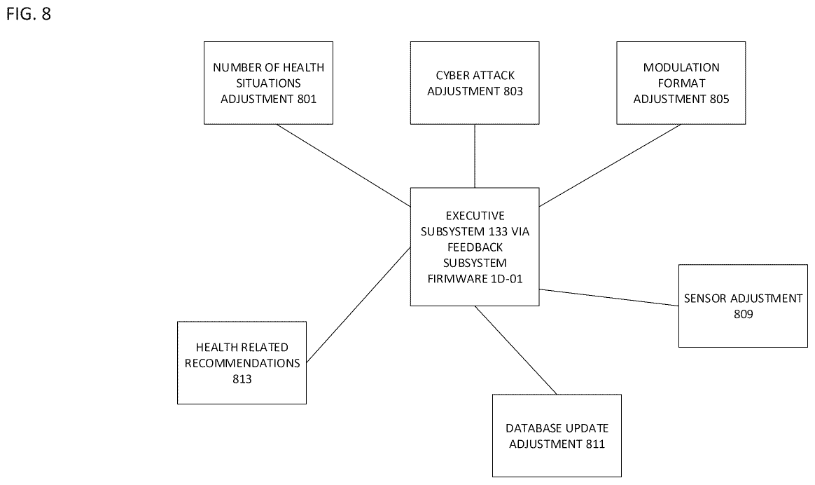

In some embodiments, the prospective actions available for selection comprise environmental actions, as previously discussed. Environmental actions are actions to adapt the intelligent healthcare transponder system 100 to varying external conditions. This comprises dynamically adjusting operational parameters which are available to executive subsystem 133 to adjust via feedback subsystem firmware 1D-01. Examples of adjustments are shown in FIG. 8, and include but are not limited to:
Healthcare capacity adjustment 801,
Cyber attack adjustment 803,
Modulator and symbol mapper adjustments 805, comprising:
   Adjustments to plurality of symbol mappers 107 such as changing number of symbol mappers and symbol mapper dimensions, and
   Adjustments to plurality of multidimensional mappers 109 such as changing number of multidimensional modulators and modulator dimensions,
Adjustments to sensors used to obtain measured signals 103,
Adjustments to the training selector module 101 to adjust, for example, selection parameters or techniques,
Adjustments to the training dataset 106, and
Adjustments for health-related recommendations.

By executing these environmental actions, the executive subsystem 133 continuously optimizes the intelligent healthcare transponder system 100 to adapt to changing conditions and deliver consistent, high-quality service.

In some embodiments, the prospective actions available for selection comprise internal commands. Internal commands are instructions sent by the executive module to the perceptor to modify modeling parameters. In some embodiments, internal commands comprise:
adjustments to the focus level,
adjustments to the precision factor vector, and
adjustments related to trade-offs between computational cost and cognitive decision-making accuracy.

In step 508, the selected prospective actions are then tested by at least one of one or more executive processing modules 2C-03-01 to 2C-03-N, and planning module 2C-13 within a virtual environment. The virtual environment simulates potential adjustments in a risk-free manner, allowing the system to evaluate the impact on an internal reward function prior to deployment in the “real-world”. By performing these simulations, an indication of the probability of success in the real world is obtained, and the probability of adverse consequences in the real-world is reduced.

The internal reward function is now explained. The internal reward function is designed to achieve one or more goals of the intelligent healthcare transponder system 100. In some embodiments, for PAC k, and symbol n, the internal reward function denoted as

$${rw}_{n}^{k} ,$$

based on the assurance factor, the incremental change in the assurance factor as explained below:

$${rw}_{n}^{k} = f ( {AF}_{n}^{k} , {Δ}_{n}^{AF , k} , {SDN}_{k}^{n} ) \qquad ( Equation 28 )$$

where:
   ƒ(⋅) is a function,

$${AF}_{n}^{k}$$

is the assurance factor for PAC K and symbol n as explained previously;

$${Δ}_{n}^{AF , k}$$

is the change in the assurance factor for PAC k compared to PAC (k−1) for symbol n, as explained previously; and

$${SDN}_{n}^{k}$$

is a set of parameters for symbol n and PAC k intended for optimization in the intelligent healthcare transponder system 100. These include the parameters for adjustment shown in FIG. 8.

In other embodiments,

$${rw}_{n}^{k}$$

is based on the estimated DE and the incremental change in DE:

$${rw}_{n}^{k} = f ( 〈 {DE}_{n}^{k} 〉 , {Δ}_{k}^{n} , {SDN}_{k}^{n} \qquad ( Equation 29 )$$

where:
   ƒ(⋅) and

$${SDN}_{n}^{k}$$

are as previously defined,

$$〈 {DE}_{n}^{k} 〉$$

is the estimated DE for PAC k and symbol n as explained previously; and

$${Δ}_{n}^{k}$$

is the change in the DE for PAC k compared to PAC (k−1) for symbol n that is;

$${Δ}_{k}^{n} = 〈 {DE}_{n}^{k - 1} 〉 - 〈 {DE}_{n}^{k} 〉 \qquad ( Equation 30 )$$

As explained above, the internal reward function is designed to achieve one or more goals of the intelligent healthcare transponder system 100. An example reward function is demonstrated below for embodiments where achieving the goals of optimizing for error and maximizing the number of diagnosable health situations is as follows:

$${rw}_{n}^{k} = \frac{〈 {DE}_{n}^{k} 〉}{( {R}_{ref} - {R}_{k} )} \qquad ( Equation 31 )$$

where:
   Rₖ is the number of health situations set for diagnosis at the current time, decremented by a fixed discretization step d, and
   R_{ref} is a reference rate determined by R_{ref}=2^{C}^{ₕₑₐₗₜₕ}.

Additionally, the planning module 2C-13 receives modeling configurations like the precision factor vector PF^{k,m} from the perceptor subsystem 143 through internal feedback channel 139, as described previously. The planning module 2C-13 then uses this information to determine the appropriate action to apply to the intelligent healthcare transponder system 100.

At least one of planning module 2C-13, and one or more executive processing modules 2C-03-01 and 2C-03-N then perform the following calculations: the ration

$${ρ}_{h}^{k + 1 , t}$$

for the next PAC (k+1) due to the virtual environmental action,

$${a}_{k + 1}^{t}$$

where t is the current virtual action index, is calculated based on the standard deviation of the observed values for current PAC k and previous PAC (k−1), that is:

$${ρ}_{h}^{k + 1 , t} = {max}_{d = 1 , 2 , … , D} ( \frac{std ( {\hat{Y}}_{n}^{k , h , m} \vert {\hat{X}}_{n}^{k , h} = {X}_{d} )}{std ( {\hat{Y}}_{n}^{k - 1 , h , m} \vert {\hat{X}}_{n}^{k - 1 , h} = {X}_{d} )} ) \qquad ( Equation 32 )$$

where D represents the maximum number of health situations in the dataset during training mode, and
   the current number of health situations during steady state mode is D=Rₖ.

In some embodiments, predicted discretized data vector for PAC (k+1), current virtual action index t and the focus level m is denoted as

$${\hat{Y}}_{n}^{( k + 1 ) , t , m} \cdot {\hat{Y}}_{n}^{( k + 1 ) , t , m}$$

is calculated as:

$${\hat{Y}}_{n}^{( k + 1 ) , t , m} = q ( {\hat{Y}}_{n}^{k , h , m} , {ρ}^{k + 1 , t} , {c}_{k} , {c}_{k - 1} , t , m ) \qquad ( Equation 33 )$$

where q(⋅) is a function that takes into account:
   the current discretized data vector

$${\hat{Y}}_{n}^{k , h , m}$$

the action aₖ for PAC k,
the previous action aₖ₋₁ for PAC (k−1),
the ratio

$${ρ}_{h}^{k + 1 , f} ,$$

the current virtual action index t, and
the focus level m.

In other embodiments, an example function q(⋅) to predict posterior probability for virtual environmental action

$${a}_{k + 1}^{t}$$

is:

$${\hat{Y}}_{n , h}^{( k + 1 ) , t , m} = {\hat{Y}}_{n , h}^{k , m} \times {( {ρ}_{h}^{k + 1 , t} )}^{\frac{( m + 1 ) ( t \times d )}{2 ( {R}_{k - 1} - {R}_{k} )}} \qquad ( Equation 34 )$$

### Then,

$${\hat{Y}}_{n , h}^{( k + 1 ) , t , m} = {\hat{Y}}_{n , h}^{k , m} \times {( {max}_{d = 1 , 2 , … , D} ( \frac{std ( {\hat{Y}}_{n}^{k , h , m} \vert {\hat{X}}_{n}^{k , h} = {X}_{d} )}{std ( {\hat{Y}}_{n}^{k - 1 , h , m} \vert {\hat{X}}_{n}^{k - 1 , h} = {X}_{d} )} ) )}^{\frac{( m + 1 ) ( t \times d )}{2 ( {R}_{k - 1} - {R}_{k} )}} \qquad ( Equation 35 )$$

where D represents the maximum number of health situations in the dataset during training mode and the current number of health situations during steady state mode is D=Rₖ;
   d is the discretization step for the health situation, for example, 1; and
   Rₖ and R₍ₖ₋₁₎ are the rates at the kth and (k−1)th PAC, respectively.

The standard deviation changes proportionally to alterations in the number of sensors, health situations, updated databases and cyber attacks. These equations provide an indication of how intelligent healthcare transponder system 100 will respond to potential future actions by simulating the outcome of these actions. The objective is to estimate how different configurations and conditions will affect the signal's characteristics, such as its standard deviation, in the context of healthcare systems.

In some embodiments, the predicted posterior probability due to the virtual action

$${a}_{k + 1}^{t}$$

is calculated by posterior sent by the perceptor subsystem 133 to the executive subsystem 143 through internal feedback 137 as:

$${b}_{k , h} ( {\hat{X}}_{n , h}^{( k + 1 ) , t} , {\hat{Y}}_{n , h}^{( k + 1 ) , t , m} ) = P ( {\hat{X}}_{n , h}^{( k + 1 ) , t} \vert {\hat{Y}}_{n , h}^{( k + 1 ) , t , m} ) \qquad ( Equation 36 )$$

Here, t ∈{1, 2, . . . , T} and T is a total number of imaginative actions that the kth posterior sent by perceptor, is still valid for predicting the T^{th} virtual action outcome. The assurance factor

$${AF}_{n}^{( k + 1 ) , t}$$

and its incremental change

$${Δ}_{k + 1 , t}^{AF , n}$$

are calculated as:

$$\begin{matrix}{AF}_{n}^{( k + 1 ) , t} = \frac{{\sum }_{b = n - L}^{n} P ( {\overline{X}}_{b}^{( k + 1 ) , t} \vert {\hat{Y}}_{b}^{( k + 1 ) , t , m} )}{L} , n - L \geq m , \\ {Δ}_{k + 1 , t}^{AF , n} = {AF}_{n}^{k + 1 , t} - {AF}_{n}^{k + 1 , t - 1}\end{matrix} \qquad ( Equation 37 )$$

The internal rewards for the desired virtual action

$${c}_{k + 1}^{t}$$

are calculated using:

$${rw}_{n}^{( k + 1 ) , t} = \{ \begin{matrix}{f}_{( k + 1 )} ( {AF}_{n}^{( k + 1 ) , t} , {Δ}_{k + 1 , t}^{AF , n} ) , & t ∈ \{ 1 , 2 , … , T \} \\ {rw}_{n}^{k} , & t = 0\end{matrix} \qquad ( Equation 38 )$$

As mentioned before, T is the total number of virtual actions. Then, the action that either minimizes or maximizes the internal reward is selected.

In embodiments where minimizing the internal reward is the goal, the action

$${c}_{k + 1}^{{t}_{opt}}$$

that yields the minimum internal reward is selected as:

$${t}_{opt} = {arg min}_{t ∈ \{ 0 , 1 , 2 , … , T \}} ( {rw}_{n}^{( k + 1 ) , t} ) \qquad ( Equation 39 )$$

In embodiments where maximizing the internal reward is the goal, the action

$${c}_{k + 1}^{{t}_{opt}}$$

that yields the maximum internal reward is selected as:

$${t}_{opt} = {arg max}_{t ∈ \{ 0 , 1 , 2 , … , T \}} \qquad ( Equation 40 )$$

Consequently, planning module 2C-13 selects the action to be applied to the environment, denoted as cₖ₊₁, based on tₒₚₜ. In some embodiments, the executive policy 2C-09 adjusts the threshold to enhance accuracy or accept higher complexity as warranted.

In step 709, the impact on the internal reward is evaluated to determine whether a proposed action is beneficial or otherwise.

When a proposed action proves beneficial in step 709, then in step 711 at least one of executive processing modules 2C-03-01 to 2C-03-N; and planning module 2C-13 communicates signals comprising the proposed action to healthcare feedback subsystem 155.

As explained previously, a detailed embodiment of healthcare feedback subsystem 155 is shown in FIG. 1H. The components and detailed operation of the components of healthcare feedback subsystem 155 are described below.

Healthcare feedback subsystem 155 comprises feedback processing module 1H-07, which comprises feedback subsystem firmware 1H-01 running on feedback subsystem processor 1H-03. Feedback subsystem firmware 1H-01 offers a more sophisticated mechanism for integration of NI into intelligent healthcare transponder system 100.

Feedback subsystem firmware 1H-01 determines adjustments; and generates updates for different blocks in the intelligent healthcare transponder system 100 such as transmission 102 and reception 104 based on received signals from executive subsystem 133.

One of ordinary skill in the art would understand that feedback subsystem processor 1H-03 is a processor which is appropriate for this task. Feedback processing module 1H-07 is communicatively coupled to feedback subsystem database 1H-05.

Then, feedback processing module 1H-07 within healthcare feedback subsystem 155, receives the signals comprising the proposed action from executive subsystem 133. Based on the received signals, the feedback subsystem firmware 1H-01 determines the adjustments necessary to implement the proposed action. In some embodiments, feedback subsystem firmware 1H-01 performs this determination based on data retrieved from feedback subsystem database 1D-05.

Feedback processing module 1H-07 implements the proposed action by transmitting signals to perform the determined adjustments to one or more components within transmission 102 or reception 104 via interconnections 1H-09. Feedback processing module 1H-07 also sends notifications, alerts and requests to parties such as clients, users and healthcare authorities via external networks 1H-19 and interconnections 1H-09. Interconnections 1H-09 is implemented using appropriate communications technologies.

The signals to effect adjustments are described below with reference to FIG. 8 where applicable:
Healthcare capacity adjustment 801,
Cyber attack adjustment 803,
Modulator and symbol mapper adjustments 805, comprising:
   Adjustments to plurality of symbol mappers 107 such as changing number of symbol mappers and symbol mapper dimensions,
   Adjustments to plurality of multidimensional mappers 109 such as changing number of multidimensional modulators and modulator dimensions,
Adjustments to sensors 809 used to obtain measured signals 103,
Adjustments to the training selector module 101 to adjust, for example, selection parameters or techniques,
Adjustments 811 to the training dataset 106, and
Adjustments 813 for health-related recommendations.

Along with adjustments, as described previously, feedback subsystem firmware 1H-01 generates commands to be sent to for example, switch 105 when intelligent healthcare transponder system 100 transitions from steady state mode into training mode and vice versa. Then, feedback processing module 1H-07 sends signals comprising these commands to these various components as is appropriate.

When no action enhances system performance in step 709, then in step 713 a revision to the focus level m is proposed.

In step 715 the planning module 2C-13 checks whether this proposed revision adheres to the complexity threshold established by the policy using the equations above. In some embodiments, this comprises comparing the complexity threshold to the available memory.

When the revised focus level is acceptable, then in step 717, planning module 2C-13 adjusts the DE threshold accordingly and communicates these changes to the perceptor subsystem 143 via internal commands transmitted over the internal feedforward channel 139. This feedback initiates another round of assessment and adaptation, refining the decision-making process at the new focus level.

Performing the above process flow enables the implementation of a continuous feedback loop, wherein: Based on the functioning and performance metrics of the intelligent healthcare transponder system 100, parameters related to transmission 102, input client data 103 and reception 104 are adjusted so as to improve the overall performance of intelligent healthcare transponder system 100.

The above also describes a perception action cycle for an intelligent healthcare transponder system 100 in an NGNLE.

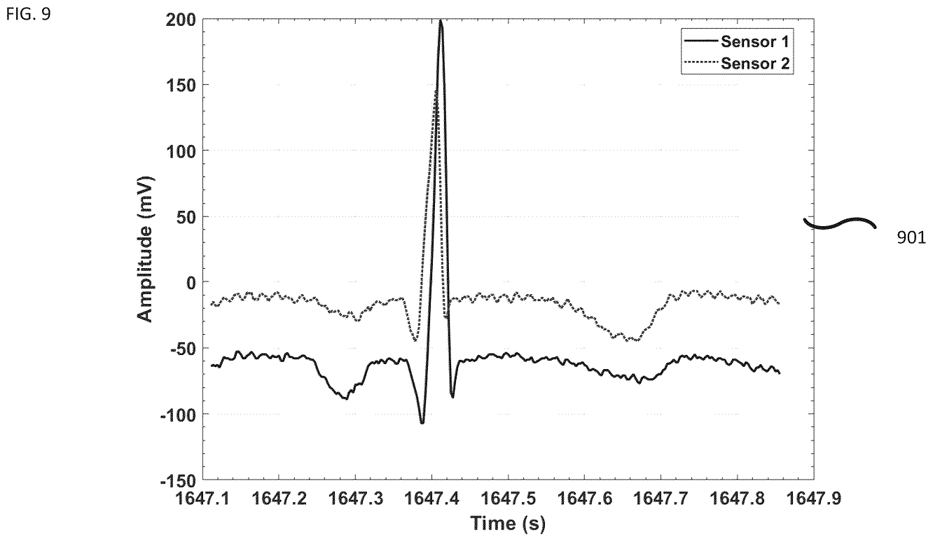

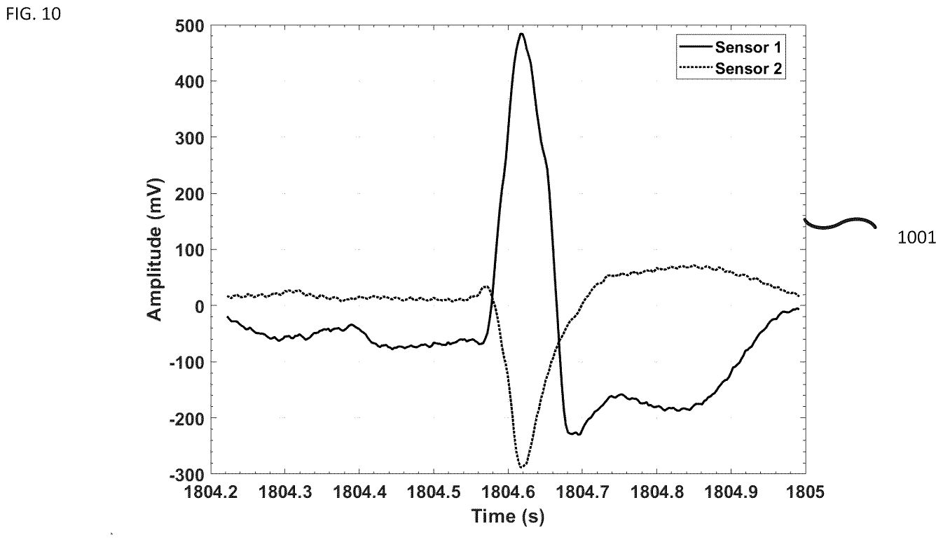

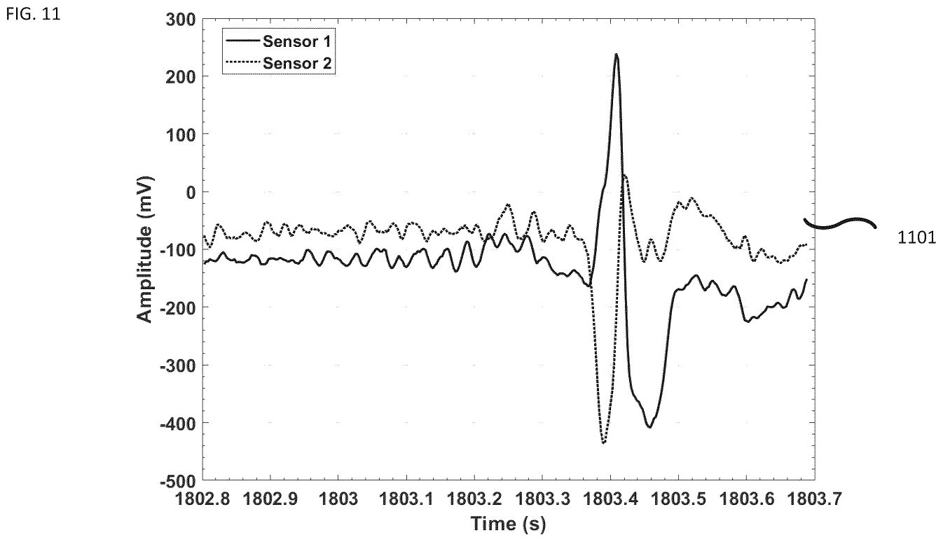

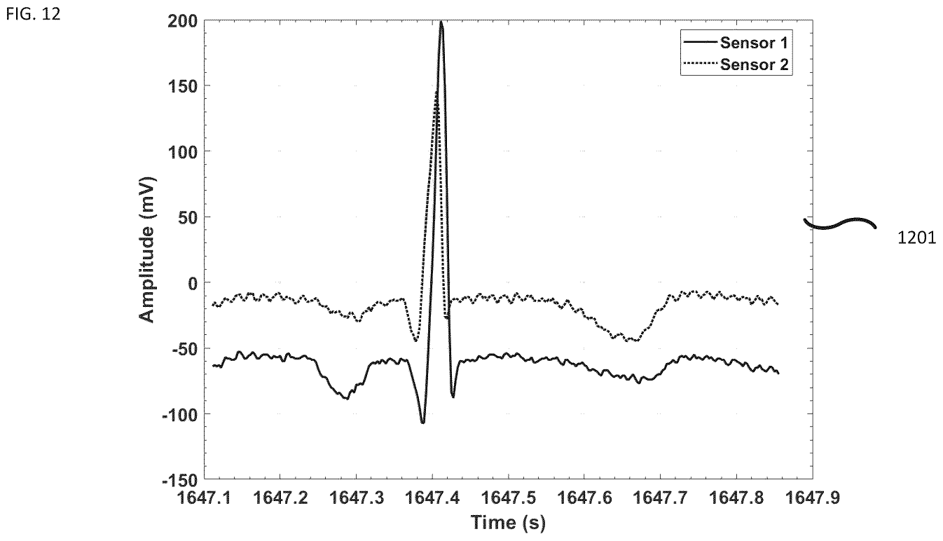

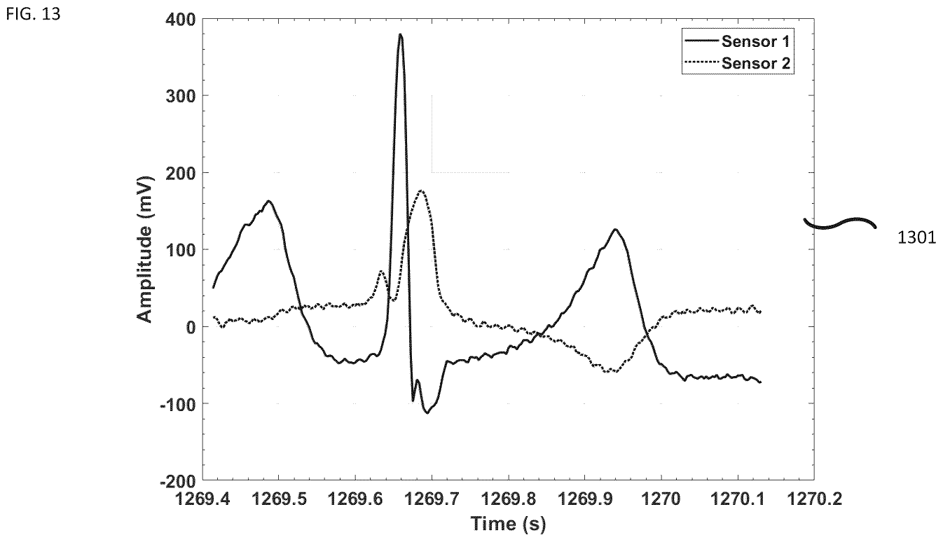

Examples of representative signals are given in FIGS. 9-13 for the MIT database as follows:
Signal 901 in FIG. 9 for h=1,
Signal 1001 in FIG. 10 for h=2,
Signal 1101 in FIG. 11 for h=3,
Signal 1201 in FIG. 12 for h=4, and
Signal 1301 in FIG. 13 for h=5.

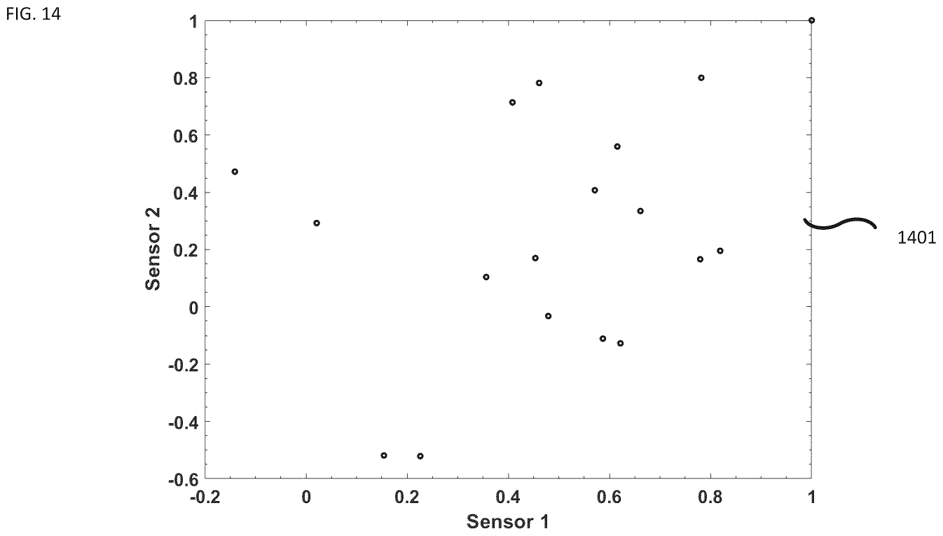

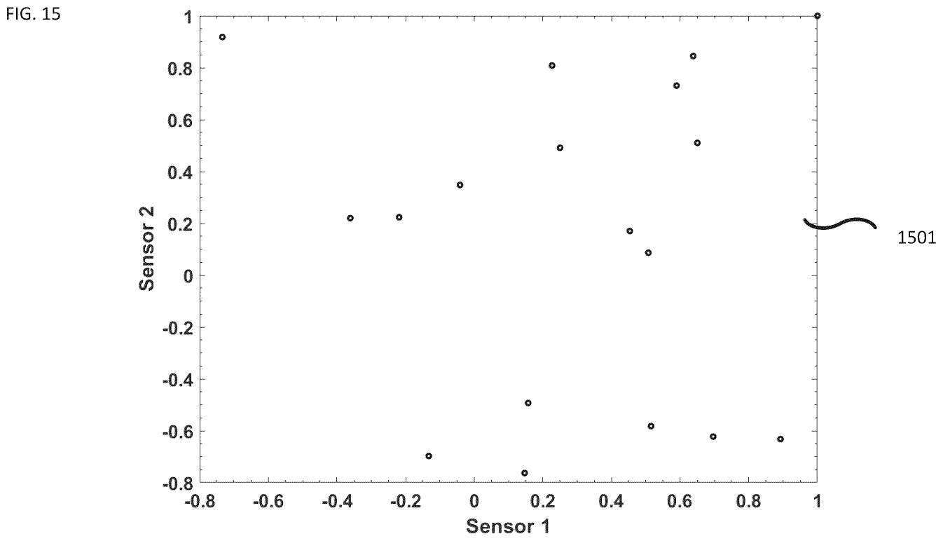

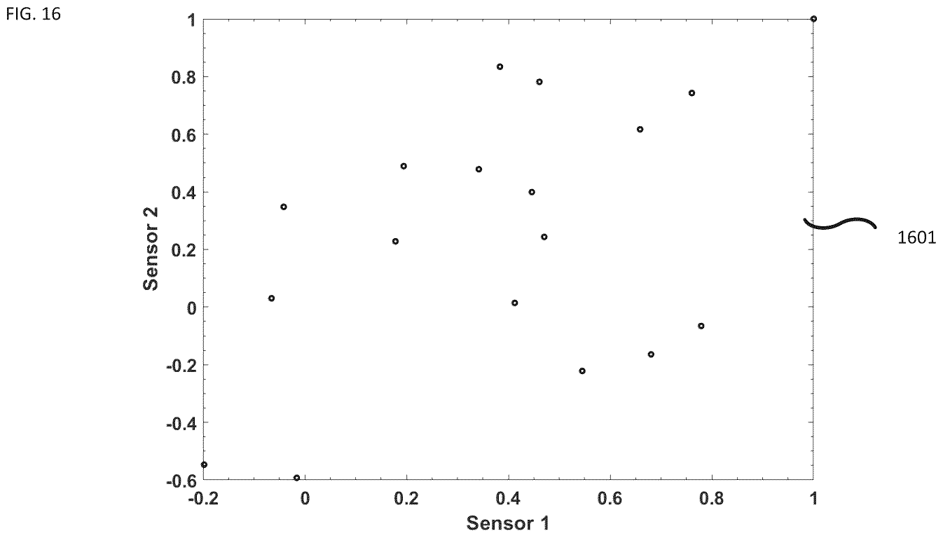

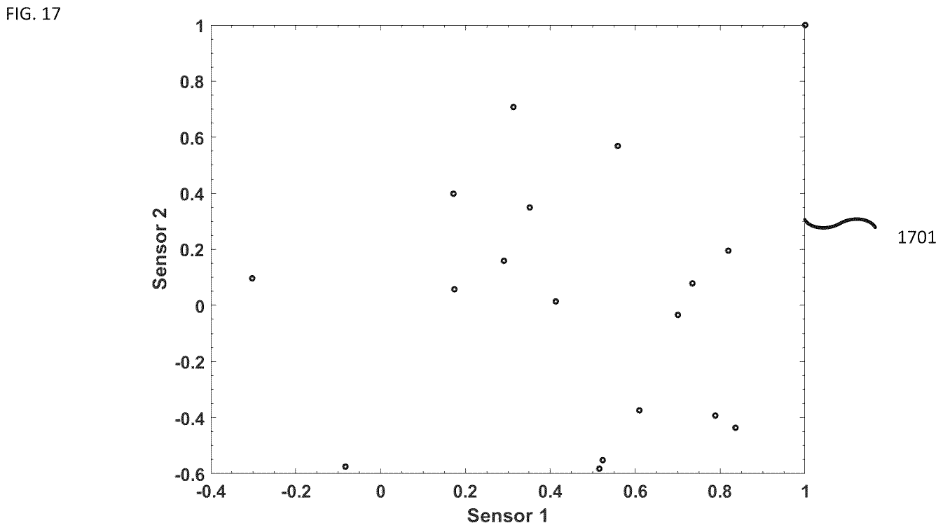

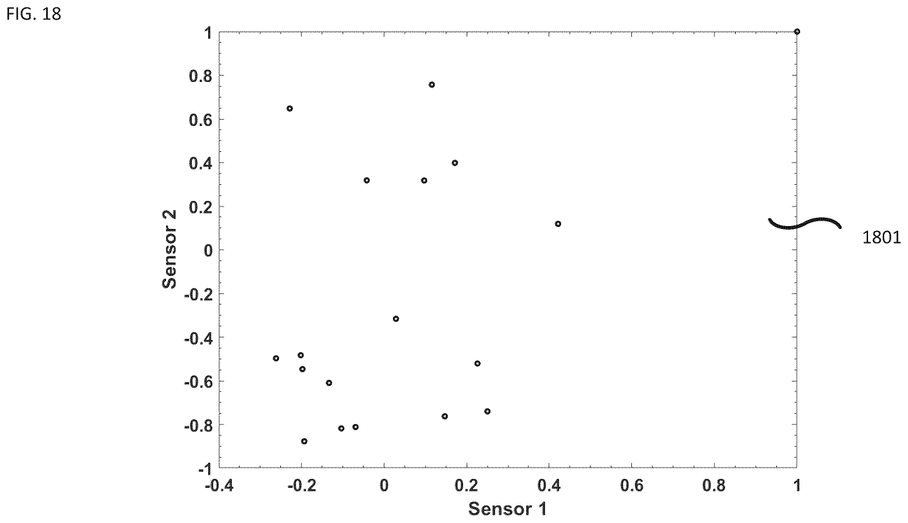

Examples of representative input symbols generated based on the representative signals are given in FIGS. 14-18 as follows:
Constellation 1401 in FIG. 14 for h=1,
Constellation 1501 in FIG. 15 for h=2,
Constellation 1601 in FIG. 16 for h=3,
Constellation 1701 in FIG. 17 for h=4, and
Constellation 1801 in FIG. 18 for h=5.

Although the algorithms described above including those with reference to the foregoing flow charts have been described separately, it should be understood that any two or more of the algorithms disclosed herein can be combined in any combination. Any of the methods, algorithms, implementations, or procedures described herein can include machine-readable instructions for execution by: (a) a processor, (b) a controller, and/or (c) any other suitable processing device. Any algorithm, software, or method disclosed herein can be embodied in software stored on a non-transitory tangible medium such as, for example, a flash memory, a CD-ROM, a floppy disk, a hard drive, a digital versatile disk (DVD), or other memory devices, but persons of ordinary skill in the art will readily appreciate that the entire algorithm and/or parts thereof could alternatively be executed by a device other than a controller and/or embodied in firmware or dedicated hardware in a well-known manner (e.g., it may be implemented by an ASIC, a programmable logic device (PLD), a field programmable logic device (FPLD), discrete logic, etc.). Also, some or all of the machine-readable instructions represented in any flowchart depicted herein can be implemented manually as opposed to automatically by a controller, processor, or similar computing device or machine. Further, although specific algorithms are described with reference to flowcharts depicted herein, persons of ordinary skill in the art will readily appreciate that many other methods of implementing the example machine readable instructions may alternatively be used. For example, the order of execution of the blocks may be changed, and/or some of the blocks described may be changed, eliminated, or combined.

It should be noted that the algorithms illustrated and discussed herein as having various modules which perform particular functions and interact with one another. It should be understood that these modules are merely segregated based on their function for the sake of description and represent computer hardware and/or executable software code which is stored on a computer-readable medium for execution on appropriate computing hardware. The various functions of the different modules and units can be combined or segregated as hardware and/or software stored on a non-transitory computer-readable medium as above as modules in any manner, and can be used separately or in combination.

While particular implementations and applications of the present disclosure have been illustrated and described, it is to be understood that the present disclosure is not limited to the precise construction and compositions disclosed herein and that various modifications, changes, and variations can be apparent from the foregoing descriptions without departing from the spirit and scope of an invention as defined in the appended claims.

What is claimed is:

1. A system for natural intelligence (NI) processing for an intelligent health transponder comprising:
a perceptor subsystem communicatively coupled to an executive subsystem via interconnections, wherein:
the perceptor subsystem comprises a posterior storage and one or more posterior processing modules coupled to each other by perceptor subsystem interconnections, and
the executive subsystem comprises an executive storage, one or more executive processing modules and a planning module coupled to each other by executive subsystem interconnections;
a feedback subsystem communicatively coupled to the executive subsystem, a transmission subsystem, a reception subsystem and a training dataset, wherein:
the feedback subsystem comprises a feedback processing module communicatively coupled to a feedback subsystem database, further wherein:
the feedback processing module comprises a feedback subsystem firmware running on a feedback subsystem processor;
an adaptive feedback path control module communicatively coupled to the executive subsystem and the perceptor subsystem, wherein:
the perceptor subsystem receives perceptions comprising a plurality of generated output symbols,
the perceptor subsystem receives a plurality of representative input symbols,
based on the received plurality of generated output symbols and plurality of representative input symbols,
the one or more posterior processing modules determining whether a suitable posterior model is available in the posterior storage,
when a suitable posterior model is available, the one or more posterior processing modules retrieves a posterior model from the posterior storage, and
the one or more posterior processing modules communicates the retrieved posterior model to the adaptive feedback path control module,
the adaptive feedback path control module estimates a diagnostic error (DE) using the retrieved posterior model,
the adaptive feedback path control module communicates the estimated DE to the executive subsystem,
when the estimated DE is below a threshold, the planning module selects a prospective action from the executive storage,
at least one of the planning module and the one or more executive processing modules test the selected prospective action in a virtual environment, at least one of the planning module and the one or more executive processing modules determines whether the selected prospective action is beneficial,
when the selected prospective action is beneficial, either the planning module or the one or more executive processing modules communicates signals comprising the selected prospective action to the feedback subsystem, and
the feedback processing module:
receives the signals comprising the selected prospective action,
determines, based on the received signals, an adjustment to implement the selected prospective action, and
transmits signals to perform the determined adjustment to one or more components within the transmission or the reception, or the training dataset.

2. The system of claim 1, wherein:
the executive subsystem comprises a healthcare calculation subsystem coupled to the executive subsystem interconnections, wherein:
the one or more posterior processing modules calculate:
a prior model,
an evidence model,
a predictive outcome model, and
a posterior model,
based on the prior model, the evidence model, the predictive outcome model and the posterior model, the healthcare calculation subsystem calculates mutual information,
based on the calculated mutual information, the healthcare calculation subsystem calculates health capacity,
based on the calculated health capacity, the healthcare calculation subsystem sets a reference rate, and
based on the reference rate, the planning module sets a diagnosis limit.

3. The system of claim 2, wherein the planning module lowers a number of health situations considered based on the calculated reference rate.

4. The system of claim 1, wherein the plurality of representative input symbols are generated using one or more representative signals selected from a training dataset.

5. The system of claim 2, wherein an alert notification of decreased capacity is sent to a user based on the calculated health capacity.

6. The system of claim 1, wherein a health situation of a client is set based on the calculated posterior model.
